SEQUENCE LISTING (214 sequences)

SEQ ID NO: 1 - protein, 58 aa, Artificial sequence
  Synthetic
ATVSDLSTKAACPAGGEAHNDKRADPAFVCRQGVVDRGRGNGCGRFGKGSIDTCAKFA

SEQ ID NO: 2 - protein, 58 aa, Artificial sequence
  Synthetic
AKLSDTKVAARCPTMGPATLAEEHQSGTVCKRDQSDRGWGNHCGLFGKGSIVTCVKAS

SEQ ID NO: 3 - protein, 58 aa, Artificial sequence
  Synthetic
ASISDMASDSRCPTQGEAYLDKQSDTQYVCKRTLVDRGWGNGCGLFGKGSLVTCAKFA

SEQ ID NO: 4 - protein, 58 aa, Artificial sequence
  Synthetic
ASISDMASDSRCPAGGEAYLDKQSDTQYVCKRTLVDRGRGNGCGRFGKGSLVTCAKFA

SEQ ID NO: 5 - protein, 14 aa, Artificial sequence
  Synthetic
DRGWGNGCGLFGKG

SEQ ID NO: 6 - protein, 18 aa, Artificial sequence
  Synthetic
RTLVDRGWGNGCGLFGKG

SEQ ID NO: 7 - protein, 18 aa, Artificial sequence
  Synthetic
DRGWGNGCGLFGKGSLVT

SEQ ID NO: 8 - protein, 22 aa, Artificial sequence
  Synthetic
RTLVDRGWGNGCGLFGKGSLVT

SEQ ID NO: 9 - protein, 32 aa, Artificial sequence
  Synthetic
QYVCKRTLVDRGWGNGCGLFGKGSLVTCAKFA

SEQ ID NO: 10 - protein, 23 aa, Artificial sequence
  Synthetic
QYVCKRTLVDRGWGNGCGLFGKG

SEQ ID NO: 11 - protein, 23 aa, Artificial sequence
  Synthetic
DRGWGNGCGLFGKGSLVTCAKFA

SEQ ID NO: 12 - protein, 14 aa, Artificial sequence
  Synthetic
DRGWGNGMGLFGKG

SEQ ID NO: 13 - protein, 18 aa, Artificial sequence
  Synthetic
RTLVDRGWGNGMGLFGKG

SEQ ID NO: 14 - protein, 18 aa, Artificial sequence
  Synthetic
DRGWGNGMGLFGKGSLVT

SEQ ID NO: 15 - protein, 22 aa, Artificial sequence
  Synthetic
RTLVDRGWGNGMGLFGKGSLVT

SEQ ID NO: 16 - protein, 14 aa, Artificial sequence
  Synthetic
DRGWGNHMGLFGKG

SEQ ID NO: 17 - protein, 18 aa, Artificial sequence
  Synthetic
RDQSDRGWGNHMGLFGKG

SEQ ID NO: 18 - protein, 18 aa, Artificial sequence
  Synthetic
DRGWGNHMGLFGKGSIVT

SEQ ID NO: 19 - protein, 22 aa, Artificial sequence
  Synthetic
RDQSDRGWGNHMGLFGKGSIVT

SEQ ID NO: 20 - protein, 18 aa, Artificial sequence
  Synthetic
QGVVDRGWGNGMGLFGKG

SEQ ID NO: 21 - protein, 18 aa, Artificial sequence
  Synthetic
DRGWGNGMGLFGKGSIDT

SEQ ID NO: 22 - protein, 22 aa, Artificial sequence
  Synthetic
QGVVDRGWGNGMGLFGKGSIDT

SEQ ID NO: 23 - protein, 18 aa, Artificial sequence
  Synthetic
HSMVDRGWGNGMGLFGKG

SEQ ID NO: 24 - protein, 18 aa, Artificial sequence
  Synthetic
DRGWGNGMGLFGKGGIVT

SEQ ID NO: 25 - protein, 22 aa, Artificial sequence
  Synthetic
HSMVDRGWGNGMGLFGKGGIVT

SEQ ID NO: 26 - protein, 14 aa, Artificial sequence
  Synthetic
DRGRGNGMGRFGKG

SEQ ID NO: 27 - protein, 18 aa, Artificial sequence
  Synthetic
QGVVDRGRGNGMGRFGKG

SEQ ID NO: 28 - protein, 18 aa, Artificial sequence
  Synthetic
DRGRGNGMGRFGKGSIDT

SEQ ID NO: 29 - protein, 22 aa, Artificial sequence
  Synthetic
QGVVDRGRGNGMGRFGKGSIDT

SEQ ID NO: 30 - protein, 20 aa, Artificial sequence
  Synthetic
MSGGTWVDVVLEHGGCVTVM

SEQ ID NO: 31 - protein, 20 aa, Artificial sequence
  Synthetic
LEHGGCVTVMAQDKPTVDIE

SEQ ID NO: 32 - protein, 20 aa, Artificial sequence
  Synthetic
AQDKPTVDIELVTTTVSNMA

SEQ ID NO: 33 - protein, 20 aa, Artificial sequence
  Synthetic
LVTTTVSNMAEVRSYCYEAS

SEQ ID NO: 34 - protein, 20 aa, Artificial sequence
  Synthetic
EVRSYCYEASISDMASDSRC

SEQ ID NO: 35 - protein, 20 aa, Artificial sequence
  Synthetic
ISDMASDSRCPTQGEAYLDK

SEQ ID NO: 36 - protein, 20 aa, Artificial sequence
  Synthetic
PTQGEAYLDKQSDTQYVCKR

SEQ ID NO: 37 - protein, 20 aa, Artificial sequence
  Synthetic
QSDTQYVCKRTLVDRGWGNG

SEQ ID NO: 38 - protein, 20 aa, Artificial sequence
  Synthetic
TLVDRGWGNGCGLFGKGSLV

SEQ ID NO: 39 - protein, 20 aa, Artificial sequence
  Synthetic
CGLFGKGSLVTCAKFACSKK

SEQ ID NO: 40 - protein, 20 aa, Artificial sequence
  Synthetic
TCAKFACSKKMTGKSIQPEN

SEQ ID NO: 41 - protein, 20 aa, Artificial sequence
  Synthetic
MTGKSIQPENLEYRIMLSVH

SEQ ID NO: 42 - protein, 20 aa, Artificial sequence
  Synthetic
LEYRIMLSVHGSQHSGMIVN

SEQ ID NO: 43 - protein, 20 aa, Artificial sequence
  Synthetic
GSQHSGMIVNDTGHETDENR

SEQ ID NO: 44 - protein, 20 aa, Artificial sequence
  Synthetic
DTGHETDENRAKVEITPNSP

SEQ ID NO: 45 - protein, 20 aa, Artificial sequence
  Synthetic
AKVEITPNSPRAEATLGGFG

SEQ ID NO: 46 - protein, 20 aa, Artificial sequence
  Synthetic
RAEATLGGFGSLGLDCEPRT

SEQ ID NO: 47 - protein, 20 aa, Artificial sequence
  Synthetic
SLGLDCEPRTGLDFSDLYYL

SEQ ID NO: 48 - protein, 20 aa, Artificial sequence
  Synthetic
GLDFSDLYYLTMNNKHWLVH

SEQ ID NO: 49 - protein, 20 aa, Artificial sequence
  Synthetic
TMNNKHWLVHKEWFHDIPLP

SEQ ID NO: 50 - protein, 20 aa, Artificial sequence
  Synthetic
KEWFHDIPLPWHAGADTGTP

SEQ ID NO: 51 - protein, 20 aa, Artificial sequence
  Synthetic
WHAGADTGTPHWNNKEALVE

SEQ ID NO: 52 - protein, 20 aa, Artificial sequence
  Synthetic
HWNNKEALVEFKDAHAKRQT

SEQ ID NO: 53 - protein, 20 aa, Artificial sequence
  Synthetic
FKDAHAKRQTVVVLGSQEGA

SEQ ID NO: 54 - protein, 20 aa, Artificial sequence
  Synthetic
VVVLGSQEGAVHTALAGALE

SEQ ID NO: 55 - protein, 20 aa, Artificial sequence
  Synthetic
VHTALAGALEAEMDGAKGRL

SEQ ID NO: 56 - protein, 20 aa, Artificial sequence
  Synthetic
AEMDGAKGRLSSGHLKCRLK

SEQ ID NO: 57 - protein, 20 aa, Artificial sequence
  Synthetic
SSGHLKCRLKMDKLRLKGVS

SEQ ID NO: 58 - protein, 20 aa, Artificial sequence
  Synthetic
MDKLRLKGVSYSLCTAAFTF

SEQ ID NO: 59 - protein, 20 aa, Artificial sequence
  Synthetic
YSLCTAAFTFTKIPAETLHG

SEQ ID NO: 60 - protein, 20 aa, Artificial sequence
  Synthetic
TKIPAETLHGTVTVEVQYAG

SEQ ID NO: 61 - protein, 20 aa, Artificial sequence
  Synthetic
TVTVEVQYAGTDGPCKVPAQ

SEQ ID NO: 62 - protein, 20 aa, Artificial sequence
  Synthetic
TDGPCKVPAQMAVDMQTLTP

SEQ ID NO: 63 - protein, 20 aa, Artificial sequence
  Synthetic
MAVDMQTLTPVGRLITANPV

SEQ ID NO: 64 - protein, 20 aa, Artificial sequence
  Synthetic
VGRLITANPVITESTENSKM

SEQ ID NO: 65 - protein, 20 aa, Artificial sequence
  Synthetic
ITESTENSKMMLELDPPFGD

SEQ ID NO: 66 - protein, 20 aa, Artificial sequence
  Synthetic
MLELDPPFGDSYIVIGVGEK

SEQ ID NO: 67 - protein, 20 aa, Artificial sequence
  Synthetic
SYIVIGVGEKKITHHWHRSG

SEQ ID NO: 68 - protein, 20 aa, Artificial sequence
  Synthetic
KITHHWHRSGSTIGKAFEAT

SEQ ID NO: 69 - protein, 20 aa, Artificial sequence
  Synthetic
STIGKAFEATVRGAKRMAVL

SEQ ID NO: 70 - protein, 20 aa, Artificial sequence
  Synthetic
VRGAKRMAVLGDTAWDFGSV

SEQ ID NO: 71 - protein, 20 aa, Artificial sequence
  Synthetic
GDTAWDFGSVGGALNSLGKG

SEQ ID NO: 72 - protein, 20 aa, Artificial sequence
  Synthetic
GGALNSLGKGIHQIFGAAFK

SEQ ID NO: 73 - protein, 20 aa, Artificial sequence
  Synthetic
IHQIFGAAFKSLFGGMSWFS

SEQ ID NO: 74 - protein, 20 aa, Artificial sequence
  Synthetic
SLFGGMSWFSQILIGTLLMW

SEQ ID NO: 75 - protein, 20 aa, Artificial sequence
  Synthetic
QILIGTLLMWLGLNTKNGSI

SEQ ID NO: 76 - protein, 19 aa, Artificial sequence
  Synthetic
LGLNTKNGSISLMCLALGG

SEQ ID NO: 77 - protein, 20 aa, Artificial sequence
  Synthetic
GSQHSGMIGYETDEDRAKVE

SEQ ID NO: 78 - protein, 20 aa, Artificial sequence
  Synthetic
MIGYETDEDRAKVEVTPNSP

SEQ ID NO: 79 - protein, 20 aa, Artificial sequence
  Synthetic
AARCPTMGPATLAEEHQSGT

SEQ ID NO: 80 - protein, 20 aa, Artificial sequence
  Synthetic
TLAEEHQSGTVCKRDQSDRG

SEQ ID NO: 81 - protein, 20 aa, Artificial sequence
  Synthetic
DVGCSVDFSKKETRCGTGVF

SEQ ID NO: 82 - protein, 20 aa, Artificial sequence
  Synthetic
KETRCGTGVFIYNDVEAWRD

SEQ ID NO: 83 - protein, 20 aa, Artificial sequence
  Synthetic
IYNDVEAWRDRYKYHPDSPR

SEQ ID NO: 84 - protein, 20 aa, Artificial sequence
  Synthetic
RYKYHPDSPRRLAAAVKQAW

SEQ ID NO: 85 - protein, 20 aa, Artificial sequence
  Synthetic
RLAAAVKQAWEEGICGISSV

SEQ ID NO: 86 - protein, 20 aa, Artificial sequence
  Synthetic
EEGICGISSVSRMENIMWKS

SEQ ID NO: 87 - protein, 20 aa, Artificial sequence
  Synthetic
SRMENIMWKSVEGELNAILE

SEQ ID NO: 88 - protein, 20 aa, Artificial sequence
  Synthetic
VEGELNAILEENGVQLTVVV

SEQ ID NO: 89 - protein, 20 aa, Artificial sequence
  Synthetic
ENGVQLTVVVGSVKNPMWRG

SEQ ID NO: 90 - protein, 20 aa, Artificial sequence
  Synthetic
GSVKNPMWRGPQRLPVPVNE

SEQ ID NO: 91 - protein, 20 aa, Artificial sequence
  Synthetic
PQRLPVPVNELPHGWKAWGK

SEQ ID NO: 92 - protein, 20 aa, Artificial sequence
  Synthetic
LPHGWKAWGKSYFVRAAKTN

SEQ ID NO: 93 - protein, 20 aa, Artificial sequence
  Synthetic
SYFVRAAKTNNSFVVDGDTL

SEQ ID NO: 94 - protein, 20 aa, Artificial sequence
  Synthetic
NSFVVDGDTLKECPLEHRAW

SEQ ID NO: 95 - protein, 20 aa, Artificial sequence
  Synthetic
KECPLEHRAWNSFLVEDHGF

SEQ ID NO: 96 - protein, 20 aa, Artificial sequence
  Synthetic
NSFLVEDHGFGVFHTSVWLK

SEQ ID NO: 97 - protein, 20 aa, Artificial sequence
  Synthetic
GVFHTSVWLKVREDYSLECD

SEQ ID NO: 98 - protein, 20 aa, Artificial sequence
  Synthetic
VREDYSLECDPAVIGTAVKG

SEQ ID NO: 99 - protein, 20 aa, Artificial sequence
  Synthetic
PAVIGTAVKGREAAHSDLGY

SEQ ID NO: 100 - protein, 20 aa, Artificial sequence
  Synthetic
REAAHSDLGYWIESEKNDTW

SEQ ID NO: 101 - protein, 20 aa, Artificial sequence
  Synthetic
WIESEKNDTWRLKRAHLIEM

SEQ ID NO: 102 - protein, 20 aa, Artificial sequence
  Synthetic
RLKRAHLIEMKTCEWPKSHT

SEQ ID NO: 103 - protein, 20 aa, Artificial sequence
  Synthetic
KTCEWPKSHTLWTDGVEESD

SEQ ID NO: 104 - protein, 20 aa, Artificial sequence
  Synthetic
LWTDGVEESDLIIPKSLAGP

SEQ ID NO: 105 - protein, 20 aa, Artificial sequence
  Synthetic
LIIPKSLAGPLSHHNTREGY

SEQ ID NO: 106 - protein, 20 aa, Artificial sequence
  Synthetic
LSHHNTREGYRTQVKGPWHS

SEQ ID NO: 107 - protein, 20 aa, Artificial sequence
  Synthetic
RTQVKGPWHSEELEIRFEEC

SEQ ID NO: 108 - protein, 20 aa, Artificial sequence
  Synthetic
EELEIRFEECPGTKVYVEET

SEQ ID NO: 109 - protein, 20 aa, Artificial sequence
  Synthetic
PGTKVYVEETCGTRGPSLRS

SEQ ID NO: 110 - protein, 20 aa, Artificial sequence
  Synthetic
CGTRGPSLRSTTASGRVIEE

SEQ ID NO: 111 - protein, 20 aa, Artificial sequence
  Synthetic
TTASGRVIEEWCCRECTMPP

SEQ ID NO: 112 - protein, 20 aa, Artificial sequence
  Synthetic
WCCRECTMPPLSFRAKDGCW

SEQ ID NO: 113 - protein, 20 aa, Artificial sequence
  Synthetic
LSFRAKDGCWYGMEIRPRKE

SEQ ID NO: 114 - protein, 20 aa, Artificial sequence
  Synthetic
YGMEIRPRKEPESNLVRSMV

SEQ ID NO: 115 - protein, 14 aa, Artificial sequence
  Synthetic
PESNLVRSMVTAGS

SEQ ID NO: 116 - protein, 20 aa, Artificial sequence
  Synthetic
SRCTHLENRDFVTGTQGTTR

SEQ ID NO: 117 - protein, 20 aa, Artificial sequence
  Synthetic
FVTGTQGTTRVTLVLELGGC

SEQ ID NO: 118 - protein, 20 aa, Artificial sequence
  Synthetic
VTLVLELGGCVTITAEGKPS

SEQ ID NO: 119 - protein, 20 aa, Artificial sequence
  Synthetic
VTITAEGKPSMDVWLDAIYQ

SEQ ID NO: 120 - protein, 20 aa, Artificial sequence
  Synthetic
MDVWLDAIYQENPAKTREYC

SEQ ID NO: 121 - protein, 20 aa, Artificial sequence
  Synthetic
ENPAKTREYCLHAKLSDTKV

SEQ ID NO: 122 - protein, 20 aa, Artificial sequence
  Synthetic
LHAKLSDTKVAARCPTMGPA

SEQ ID NO: 123 - protein, 20 aa, Artificial sequence
  Synthetic
AARCPTMGPATLAEEHQGGT

SEQ ID NO: 124 - protein, 20 aa, Artificial sequence
  Synthetic
TLAEEHQGGTVCKRDQSDRG

SEQ ID NO: 125 - protein, 20 aa, Artificial sequence
  Synthetic
VCKRDQSDRGWGNHCGLFGK

SEQ ID NO: 126 - protein, 20 aa, Artificial sequence
  Synthetic
WGNHCGLFGKGSIVACVKAA

SEQ ID NO: 127 - protein, 20 aa, Artificial sequence
  Synthetic
GSIVACVKAACEAKKKATGH

SEQ ID NO: 128 - protein, 20 aa, Artificial sequence
  Synthetic
CEAKKKATGHVYDANKIVYT

SEQ ID NO: 129 - protein, 20 aa, Artificial sequence
  Synthetic
VYDANKIVYTVKVEPHTGDY

SEQ ID NO: 130 - protein, 20 aa, Artificial sequence
  Synthetic
VKVEPHTGDYVAANETHSGR

SEQ ID NO: 131 - protein, 20 aa, Artificial sequence
  Synthetic
VAANETHSGRKTASFTISSE

SEQ ID NO: 132 - protein, 20 aa, Artificial sequence
  Synthetic
KTASFTISSEKTILTMGEYG

SEQ ID NO: 133 - protein, 20 aa, Artificial sequence
  Synthetic
KTILTMGEYGDVSLLCRVAS

SEQ ID NO: 134 - protein, 20 aa, Artificial sequence
  Synthetic
DVSLLCRVASGVDLAQTVIL

SEQ ID NO: 135 - protein, 20 aa, Artificial sequence
  Synthetic
GVDLAQTVILELDKTVEHLP

SEQ ID NO: 136 - protein, 20 aa, Artificial sequence
  Synthetic
ELDKTVEHLPTAWQVHRDWF

SEQ ID NO: 137 - protein, 20 aa, Artificial sequence
  Synthetic
TAWQVHRDWFNDLALPWKHE

SEQ ID NO: 138 - protein, 20 aa, Artificial sequence
  Synthetic
NDLALPWKHEGAQNWNNAER

SEQ ID NO: 139 - protein, 20 aa, Artificial sequence
  Synthetic
GAQNWNNAERLVEFGAPHAV

SEQ ID NO: 140 - protein, 20 aa, Artificial sequence
  Synthetic
LVEFGAPHAVKMDVYNLGDQ

SEQ ID NO: 141 - protein, 20 aa, Artificial sequence
  Synthetic
KMDVYNLGDQTGVLLKALAG

SEQ ID NO: 142 - protein, 20 aa, Artificial sequence
  Synthetic
TGVLLKALAGVPVAHIEGTK

SEQ ID NO: 143 - protein, 20 aa, Artificial sequence
  Synthetic
VPVAHIEGTKYHLKSGHVTC

SEQ ID NO: 144 - protein, 20 aa, Artificial sequence
  Synthetic
YHLKSGHVTCEVGLEKLKMK

SEQ ID NO: 145 - protein, 20 aa, Artificial sequence
  Synthetic
EVGLEKLKMKGLTYTMCDKT

SEQ ID NO: 146 - protein, 20 aa, Artificial sequence
  Synthetic
GLTYTMCDKTKFTWKRAPTD

SEQ ID NO: 147 - protein, 20 aa, Artificial sequence
  Synthetic
KFTWKRAPTDSGHDTVVMEV

SEQ ID NO: 148 - protein, 20 aa, Artificial sequence
  Synthetic
SGHDTVVMEVTFSGTKPCRI

SEQ ID NO: 149 - protein, 20 aa, Artificial sequence
  Synthetic
TFSGTKPCRIPVRAVAHGSP

SEQ ID NO: 150 - protein, 20 aa, Artificial sequence
  Synthetic
PVRAVAHGSPDVNVAMLITP

SEQ ID NO: 151 - protein, 20 aa, Artificial sequence
  Synthetic
DVNVAMLITPNPTIENNGGG

SEQ ID NO: 152 - protein, 20 aa, Artificial sequence
  Synthetic
NPTIENNGGGFIEMQLPPGD

SEQ ID NO: 153 - protein, 20 aa, Artificial sequence
  Synthetic
FIEMQLPPGDNIIYVGELSH

SEQ ID NO: 154 - protein, 20 aa, Artificial sequence
  Synthetic
NIIYVGELSHQWFQKGSSIG

SEQ ID NO: 155 - protein, 20 aa, Artificial sequence
  Synthetic
QWFQKGSSIGRVFQKTKKGI

SEQ ID NO: 156 - protein, 20 aa, Artificial sequence
  Synthetic
RVFQKTKKGIERLTVIGEHA

SEQ ID NO: 157 - protein, 20 aa, Artificial sequence
  Synthetic
ERLTVIGEHAWDFGSAGGFL

SEQ ID NO: 158 - protein, 20 aa, Artificial sequence
  Synthetic
WDFGSAGGFLSSIGKAVHTV

SEQ ID NO: 159 - protein, 20 aa, Artificial sequence
  Synthetic
SSIGKAVHTVLGGAFNSIFG

SEQ ID NO: 160 - protein, 20 aa, Artificial sequence
  Synthetic
LGGAFNSIFGGVGFLPKLLL

SEQ ID NO: 161 - protein, 20 aa, Artificial sequence
  Synthetic
GVGFLPKLLLGVALAWLGLN

SEQ ID NO: 162 - protein, 20 aa, Artificial sequence
  Synthetic
GVALAWLGLNMRNPTMSMSF

SEQ ID NO: 163 - protein, 20 aa, Artificial sequence
  Synthetic
MRNPTMSMSFLLAGGLVLAM

SEQ ID NO: 164 - protein, 16 aa, Artificial sequence
  Synthetic
LLAGGLVLAMTLGVGA

SEQ ID NO: 165 - protein, 20 aa, Artificial sequence
  Synthetic
DVGCAVDTERMELRCGEGLV

SEQ ID NO: 166 - protein, 20 aa, Artificial sequence
  Synthetic
MELRCGEGLVVWREVSEWYD

SEQ ID NO: 167 - protein, 20 aa, Artificial sequence
  Synthetic
VWREVSEWYDNYAYYPETPG

SEQ ID NO: 168 - protein, 20 aa, Artificial sequence
  Synthetic
NYAYYPETPGALASAIKETF

SEQ ID NO: 169 - protein, 20 aa, Artificial sequence
  Synthetic
ALASAIKETFEEGSCGVVPQ

SEQ ID NO: 170 - protein, 20 aa, Artificial sequence
  Synthetic
EEGSCGVVPQNRLEMAMWRS

SEQ ID NO: 171 - protein, 20 aa, Artificial sequence
  Synthetic
NRLEMAMWRSSVTELNLALA

SEQ ID NO: 172 - protein, 20 aa, Artificial sequence
  Synthetic
SVTELNLALAEGEANLTVVV

SEQ ID NO: 173 - protein, 20 aa, Artificial sequence
  Synthetic
EGEANLTVVVDKFDPTDYRG

SEQ ID NO: 174 - protein, 20 aa, Artificial sequence
  Synthetic
DKFDPTDYRGGVPGLLKKGK

SEQ ID NO: 175 - protein, 20 aa, Artificial sequence
  Synthetic
GVPGLLKKGKDIKVSWKSWG

SEQ ID NO: 176 - protein, 20 aa, Artificial sequence
  Synthetic
DIKVSWKSWGHSMIWSIPEA

SEQ ID NO: 177 - protein, 20 aa, Artificial sequence
  Synthetic
HSMIWSIPEAPRRFMVGTEG

SEQ ID NO: 178 - protein, 20 aa, Artificial sequence
  Synthetic
PRRFMVGTEGQSECPLERRK

SEQ ID NO: 179 - protein, 20 aa, Artificial sequence
  Synthetic
QSECPLERRKTGVFTVAEFG

SEQ ID NO: 180 - protein, 20 aa, Artificial sequence
  Synthetic
TGVFTVAEFGVGLRTKVFLD

SEQ ID NO: 181 - protein, 20 aa, Artificial sequence
  Synthetic
VGLRTKVFLDFRQEPTHECD

SEQ ID NO: 182 - protein, 20 aa, Artificial sequence
  Synthetic
FRQEPTHECDTGVMGAAVKN

SEQ ID NO: 183 - protein, 20 aa, Artificial sequence
  Synthetic
TGVMGAAVKNGMAIHTDQSL

SEQ ID NO: 184 - protein, 20 aa, Artificial sequence
  Synthetic
GMAIHTDQSLWMRSMKNDTG

SEQ ID NO: 185 - protein, 20 aa, Artificial sequence
  Synthetic
WMRSMKNDTGTYIVELLVTD

SEQ ID NO: 186 - protein, 20 aa, Artificial sequence
  Synthetic
TYIVELLVTDLRNCSWPASH

SEQ ID NO: 187 - protein, 20 aa, Artificial sequence
  Synthetic
LRNCSWPASHTIDNADVVDS

SEQ ID NO: 188 - protein, 20 aa, Artificial sequence
  Synthetic
TIDNADVVDSELFLPASLAG

SEQ ID NO: 189 - protein, 20 aa, Artificial sequence
  Synthetic
ELFLPASLAGPRSWYNRIPG

SEQ ID NO: 190 - protein, 20 aa, Artificial sequence
  Synthetic
PRSWYNRIPGYSEQVKGPWK

SEQ ID NO: 191 - protein, 20 aa, Artificial sequence
  Synthetic
YSEQVKGPWKYTPIRVIREE

SEQ ID NO: 192 - protein, 20 aa, Artificial sequence
  Synthetic
YTPIRVIREECPGTTVTINA

SEQ ID NO: 193 - protein, 20 aa, Artificial sequence
  Synthetic
CPGTTVTINAKCDKRGASVR

SEQ ID NO: 194 - protein, 20 aa, Artificial sequence
  Synthetic
KCDKRGASVRSTTESGKVIP

SEQ ID NO: 195 - protein, 20 aa, Artificial sequence
  Synthetic
STTESGKVIPEWCCRACTMP

SEQ ID NO: 196 - protein, 20 aa, Artificial sequence
  Synthetic
EWCCRACTMPPVTFRTGTDC

SEQ ID NO: 197 - protein, 20 aa, Artificial sequence
  Synthetic
PVTFRTGTDCWYAMEIRPVH

SEQ ID NO: 198 - protein, 20 aa, Artificial sequence
  Synthetic
WYAMEIRPVHDQGGLVRSMV

SEQ ID NO: 199 - protein, 12 aa, Artificial sequence
  Synthetic
DQGGLVRSMVVA

SEQ ID NO: 200 - protein, 14 aa, Artificial sequence
  Synthetic
DRGWGNHCGLFGKG

SEQ ID NO: 201 - protein, 18 aa, Artificial sequence
  Synthetic
RDQSDRGWGNHCGLFGKG

SEQ ID NO: 202 - protein, 18 aa, Artificial sequence
  Synthetic
DRGWGNHCGLFGKGSIVT

SEQ ID NO: 203 - protein, 22 aa, Artificial sequence
  Synthetic
RDQSDRGWGNHCGLFGKGSIVT

SEQ ID NO: 204 - protein, 18 aa, Artificial sequence
  Synthetic
QGVVDRGWGNGCGLFGKG

SEQ ID NO: 205 - protein, 18 aa, Artificial sequence
  Synthetic
DRGWGNGCGLFGKGSIDT

SEQ ID NO: 206 - protein, 22 aa, Artificial sequence
  Synthetic
QGVVDRGWGNGCGLFGKGSIDT

SEQ ID NO: 207 - protein, 18 aa, Artificial sequence
  Synthetic
HSMVDRGWGNGCGLFGKG

SEQ ID NO: 208 - protein, 18 aa, Artificial sequence
  Synthetic
DRGWGNGCGLFGKGGIVT

SEQ ID NO: 209 - protein, 22 aa, Artificial sequence
  Synthetic
HSMVDRGWGNGCGLFGKGGIVT

SEQ ID NO: 210 - protein, 14 aa, Artificial sequence
  Synthetic
DRGRGNGCGRFGKG

SEQ ID NO: 211 - protein, 18 aa, Artificial sequence
  Synthetic
QGVVDRGRGNGCGRFGKG

SEQ ID NO: 212 - protein, 18 aa, Artificial sequence
  Synthetic
DRGRGNGCGRFGKGSIDT

SEQ ID NO: 213 - protein, 22 aa, Artificial sequence
  Synthetic
QGVVDRGRGNGCGRFGKGSIDT

SEQ ID NO: 214 - protein, 6 aa, Artificial sequence
  Synthetic
HHHHHH

(19) United States
(12) Patent Application Publication
(10) Pub. No.: US 2022/0341942 A1
(43) Pub. Date: Oct. 27, 2022

(54) FLAVIVIRUS ARRAYS AND USE THEREOF

(71) Applicant: NATIONAL INSTITUTE FOR BIOTECHNOLOGY IN THE NEGEV LTD., Beer-Sheva (IL)

(72) Inventors: Tomer HERTZ, Omer (IL); Lilach FRIEDMAN, Rishon Le Zion (IL)

(21) Appl. No.: 17/835,235

(22) Filed: Jun. 8, 2022

Related U.S. Application Data
(63) Continuation of application No. PCT/IL2020/051266, filed on Dec. 8, 2020.
(60) Provisional application No. 62/945,178, filed on Dec. 8, 2019.
(60) Provisional application No. 62/945,179, filed on Dec. 8, 2019.

(51) Int. Cl. G01N 33/68; G01N 33/569; G01N 33/543

(52) U.S. Cl. CPC G01N 33/6854; G01N 33/56983; G01N 33/54306; G01N 2333/185; G01N 2469/20; G01N 2800/50

(57) ABSTRACT

Arrays comprising probes comprising peptides from more than one flavivirus are provided. Methods of using the arrays, as well as kits and systems comprising the arrays are also provided.

### CROSS REFERENCE TO RELATED APPLICATIONS

This application is a bypass continuation of PCT Patent Application No. PCT/IL2020/051266 having International filing date of Dec. 8, 2020, which claims the benefit of priority of U.S. Provisional Patent Application Nos. 62/945,179, filed Dec. 8, 2019, and No. 62/945,178, filed Dec. 8, 2019, the contents of which are all incorporated herein by reference in their entirety.

### FIELD OF INVENTION

The present invention is in the field of flavivirus vaccination and diagnosis and array design.

### BACKGROUND OF THE INVENTION

The adaptive immune system provides protection against previously encountered pathogens via memory T cells and B cells. Vaccination, the most cost-effective public health intervention, stimulates the immune system to generate protective memory responses. A variety of factors impact an individual's heterogeneity in vaccine induced immune responses, such as age and gender. One important and understudied factor is ‘immunological history’—the individual's memory antibody repertoire to previously encountered pathogens and vaccines. This lack of knowledge is due to the lack of a systematic approach to quantify immunological history and study its effects.

During secondary flavivirus infection this memory response can have an unintended negative effect. Antibody dependent enhancement (ADE) of flavivirus infection was first discovered in Dengue virus infections. This enhancement results in increased severity of the disease following a secondary infection with a different Dengue subtype. Antibodies generated during the first infection are not sufficient (due to concentration or avidity) to neutralize the second infection by another subtype. These non-optimal antibodies, instead of helping fight infection actually increase the severity of the second infection. One hypothesis as to the mechanism of ADE is that the antibodies from the first infection may opsonize the secondary virus and target it for Fc-receptor-mediated endocytosis into monocytes and macrophages, which are the principal sites of Dengue viral replication. This drives a higher viral load and worse symptoms.

The Dengue virus vaccine Dengvaxia, is only recommended for subjects that have a previous confirmed Dengue infection. This is due to the fact that in naive subjects the immune response caused by Dengvaxia, increases the risk of ADE upon infection. Unfortunately, there is currently no reliable way to determine prior dengue virus infection. Further, there is no known method to predict which subjects will develop ADE after a first infection or a vaccination. Even more there is no known way to predict which vaccines will cause increased risk of ADE and which will not.

The problem is even more widespread, as it has been shown that prior vaccination or infection with one flavivirus can lead to ADE upon infection with a different flavivirus. Further, ADE is not limited to Dengue but has been reported in other flaviviruses as well. Thus, for example vaccination or infection by a first flavivirus, such as West Nile Virus, can lead to ADE after infection with a second flavivirus, such as Zika. In Zika in particular, development of ADE can have disastrous neurological effects, in particular on fetuses. Since many flaviviruses are regularly vaccinated against (such as Tick-borne encephalitis for example), a large portion of the world population may be at risk for developing ADE.

A simple, rapid, highly sensitive assay to asses the risk of developing ADE upon flavivirus infection, that requires a small amount of sample and can be standardized and run at point of care locations is greatly needed. Further, a method of determining previous flavivirus infection is also needed. Lastly, a method for testing new vaccines for their risk of causing ADE is of great importance.

### SUMMARY OF THE INVENTION

The present invention provides arrays comprising probes from more than one flavivirus. Methods of using the arrays of the invention, as well as kits and systems comprising the arrays are also provided.

According to a first aspect, there is provided an array comprising a plurality of probes each immobilized at a discrete location on the array, wherein the plurality of probes comprises a probe from a first flavivirus and a probe from a second flavivirus.

According to another aspect, there is provided a method of measuring cross-reactive antibodies to a flavivirus in a subject in need thereof, the method comprising
a. providing a biological sample from the subject comprising antibodies;
b. contacting the sample to an array of the invention in conditions sufficient for antibody binding to the probes; and
c. detecting the binding of the antibodies to discrete locations on the array indicating the presence in the sample of antibodies to probes located at the detected discrete locations;

thereby measuring cross-reactive antibodies to a flavivirus.

According to another aspect, there is provided a method of predicting a risk of future ADE induction due to vaccination by a flavivirus vaccine, the method comprising:
a. providing a solution comprising antibodies from immune cells contacted by the flavivirus vaccine;
b. contacting the solution to an array of the invention in conditions sufficient for antibody binding to the probes;
c. detecting the binding of the antibodies to discrete locations on the array indicating the presence in the solution of antibodies to probes located at the detected discrete locations; and
d. generating a flavivirus immune score from the detected binding, wherein the magnitude of the flavivirus immune score is proportional to the risk of future ADE induction due to vaccination by the flavivirus vaccine;

thereby predicting the risk of future ADE induction due to vaccination by a flavivirus vaccine.

According to another aspect, there is provided a kit comprising an array of the invention, and a labeled secondary antibody configured for detection of antibodies bound to the array.

According to another aspect, there is provided a system comprising an array of the invention, and a detector configured to detect binding of antibodies to probes immobilized on the array.

According to some embodiments, the array comprises at least two probes from each flavivirus.

According to some embodiments, the flavivirus is selected from the group consisting of: Zika virus, Dengue virus type 1, Dengue virus type 2, Dengue virus type 3, Dengue virus type 4, West Nile virus, Japanese encephalitis virus, Tick-borne encephalitis virus, Louping ill virus, Omsk hemorrhagic fever virus, Powassan virus, Apoi virus, Yokose virus, Yellow fever virus, Rocio virus, Ilheus Virus, Bagaza virus, St. Louis encephalitis virus, Murray Valley encephalitis virus, Alfuy Virus and Usutu virus.

According to some embodiments, the flavivirus is selected from the group consisting of: Zika virus, Dengue virus type 1, Dengue virus type 2, Dengue virus type 3, Dengue virus type 4, West Nile virus, Japanese encephalitis virus, and Tick-borne encephalitis virus.

According to some embodiments, the first and second flaviviruses are Zika virus and Tick-borne encephalitis virus.

According to some embodiments, the first and second flaviviruses are selected from Dengue virus type 1, Dengue virus type 2, Dengue virus type 3, and Dengue virus type 4.

According to some embodiments, the probes are selected from a whole virus, a lysed virus, a virus-like particle (VLP), a whole recombinant protein and a peptide.

According to some embodiments, the plurality of probes comprises a peptide probe from each of the flaviviruses.

According to some embodiments, the plurality of probes comprises a peptide probe from a viral envelope protein from each of the flaviviruses.

According to some embodiments, the plurality of probes comprises a peptide probe from a viral NS1 protein from each of the flaviviruses.

According to some embodiments, the peptide probe comprises between 10 and 60 consecutive amino acids from a flavivirus protein.

According to some embodiments, the peptide probe comprises a fusion loop region from each of the flaviviruses.

According to some embodiments, the peptide probe comprises a recombinant protein from a flavivirus.

According to some embodiments, a probe comprising a peptide is mutated to replace a cysteine residue with a methionine residue.

According to some embodiments, the plurality of probes comprises an inactivated form of each of the flavivirus.

According to some embodiments, the plurality of probes comprises a virus-like particle (VLP) of each of the flaviviruses.

According to some embodiments, the plurality of probes further comprises lysate from a cell infected by each of the flavivirus.

According to some embodiments, the array of the invention comprises serial dilutions of at least one probe, wherein each dilution is immobilized at a discrete location on the array.

According to some embodiments, the plurality of probes is selected from Table 1, Table 2 or both.

According to some embodiments, the array of the invention is for use in determining the presence of cross-reactive antibodies to a flavivirus in a sample.

According to some embodiments, the array of the invention is for use in assessing the risk of a subject developing antibody dependent enhancement (ADE) upon infection of the subject with a flavivirus.

According to some embodiments, the ADE is Zika ADE or Dengue ADE.

According to some embodiments, the array of the invention is for use in detecting previous flavivirus infection or vaccination of a subject.

According to some embodiments, the array of the invention is for use in determining the flavivirus that had previously infected or been vaccinated against in the subject.

According to some embodiments, the subject has previously been vaccinated against a flavivirus or previously been infected by a flavivirus.

According to some embodiments, the flavivirus is selected from the group consisting of: Zika virus, Dengue virus type 1, Dengue virus type 2, Dengue virus type 3, Dengue virus type 4, West Nile virus, Japanese encephalitis virus, Tick-borne encephalitis virus, Louping ill virus, Omsk hemorrhagic fever virus, Powassan virus, Apoi virus, Yokose virus, Yellow fever virus, Rocio virus, Ilheus Virus, Bagaza virus, St. Louis encephalitis virus, Murray Valley encephalitis virus, Alfuy Virus and Usutu virus.

According to some embodiments, the biological sample is a peripheral blood sample, a plasma sample or a serum sample.

According to some embodiments, the detecting comprises contacting the array with bound antibodies with labeled secondary antibodies against the antibodies in the biological sample.

According to some embodiments, the detecting further comprises scanning the array with a detector configured to detect the labeled secondary antibodies and producing an output of the discrete locations where antibody was detected.

According to some embodiments, the method is a method of assessing a risk of developing ADE upon infection of the subject by a flavivirus, and further comprising:
d. generating a flavivirus immune score from said detected binding, wherein the magnitude of said immune score is proportional to the risk of developing ADE.

According to some embodiments, a higher immune score indicates a greater risk of developing ADE upon flavivirus infection, and wherein a lower immune score indicates a lesser risk of developing ADE upon flavivirus infection.

According to some embodiments, an immune score above a predetermined threshold indicates the subject is at an increased risk of developing ADE upon flavivirus infection.

According to some embodiments, the detector is configured to detect labeled secondary antibodies.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B: (1A) A line graph of ADE in groups of subjects at various time points after receiving TBE vaccination (Swiss cohort). (1B) A bar graph of IgG levels to lysed Zika virus, as measured by ELISA for selected subjects. Subjects are divided into those with high ADE, low ADE, and no ADE. IgG levels do not always correlate with ADE.

FIG. 2: Line graph of antibody binding to the probes of the array of the invention for the Siberian cohort.

FIGS. 3A-3B: (3A) A schematic of the 58 amino acid sequences of the fusion loops from Zika and TBE viruses. The 14-amino acid peptide used in 3B is shown in a black box. (3B) Box and whisker plots of individual's MFI for the 58 amino acid Zika fusion loop probe (left; Seq ID No 3 in Table 2) and the 14-amino acid fragment from the Zika fusion loop probe (right; Seq ID No 5 in Table 2). Results are from the Siberian cohort.

FIGS. 4A-4G: Box and whisker plots of antibody binding to whole virus probes from (4A) TBE vaccine strain, (4B) Dengue type 2, (4C) Dengue type 3, (4D) Dengue type 1, (4E) Dengue type 4, (4F) Zika virus, and (4G) Yellow Fever virus. Results are from the Swiss cohort.

FIG. 5: Schematic diagram of one block from the array of the invention. The array implementation may contain 1 or more blocks, depending on the number of antigens (and viruses) covered.

FIGS. 6A-6B: Box and whisker plots of antibody binding to probes that are (6A) recombinant Dengue type 1 NS1 protein, and (6B) recombinant Zika Envelope (ENV) protein. Results are from the Swiss cohort.

FIGS. 7A-7D: Box and whisker plots of antibody binding to peptides that are (7A) the complete Zika fusion loop (58 aa), (7B) amino acids 3-22 of the Zika fusion loop, (7C) the C-terminal 32 amino acids of the Zika fusion loop and (7D) a mutated non-functional version of the complete Zika fusion loop. Results are from the Swiss cohort.

### DETAILED DESCRIPTION OF THE INVENTION

The present invention, in some embodiments, provides arrays comprising a plurality of probes, each probe is immobilized at a discrete location on the array, and the plurality of probes comprises a probe from a first flavivirus and a probe from a second flavivirus. Kits and systems comprising the arrays of the invention are also provided, as are methods of using the arrays, kits and systems of the invention for determining the presence of cross-reactive antibodies to a flavivirus in a sample, assessing the risk of developing antibody dependent enhancement (ADE) upon infection or vaccination with a flavivirus, or detecting previous flavivirus infection or vaccination of a subject.

The present invention is based on the surprising finding that flavivirus immune-history antibody profiles correlate with the risk of future ADE development. That is, by assaying antibody binding to specific peptides, proteins and whole virus preparations from various flaviviruses, a flavivirus immune score can be generated for an individual or a vaccine. This immune score can predict the likelihood of future ADE development upon infection or vaccination with a flavivirus. Further, the likelihood of developing ADE for particular flaviviruses can be predicted.

The use of an array of probes allows for a quick and accurate assessment of the flavivirus immune history of a subject. Further, the array only requires a small amount of sample from an individual. This way, a single blood draw can be used to evaluate a subject flavivirus history, across multiple diseases simultaneously.

By a first aspect, there is provided a solid support comprising a plurality of probes, wherein the plurality of probes comprises a probe from a first flavivirus and a probe from a second flavivirus.

By a first aspect, there is provided an array comprising a plurality of probes, wherein the plurality of probes comprises a probe from a first flavivirus and a probe from a second flavivirus.

In some embodiments, the solid support is an array. In some embodiments, the solid support is a chip. As used herein, the term “array” refers to a solid support with regularly spaced probes attached to distinct and defined locations. In some embodiments, an array is an array of probes. In some embodiments, the support or array comprises probes at known locations. Thus, the location of each probe is known and so binding to a given probe can be correlated to the probe itself based on its position on the support or array. In some embodiments, an array is a single solid support with probes arrayed thereupon. In some embodiments, an array is a plurality of solid supports with probes arrayed thereupon. In some embodiments, each probe is on a separate solid support. In some embodiments, an array is an array of beads. In some embodiments, an array is an array of solid supports. Methods of making arrays and in particular protein and peptide arrays are well known in the art. Any method of making an array such as described herein may be employed. One such method is provided hereinbelow in the Materials and Methods section. Non-limiting examples of methods of producing protein/peptide arrays include U.S. Pat. No. 5,143,854, U.S. Patent Application Publication Nos. 2007/0154946, 2007/0122841, 2007/0122842, and 2008/0108149 and International Patent Application Publication No. WO/2000/003307.

The solid support, or support, refers to a material or group of materials having a rigid or semi-rigid surface or surfaces. In some embodiments, at least one surface of the solid support will be substantially flat, although in some embodiments it may be desirable to physically separate synthesis regions for different molecules with, for example, wells, raised regions, beads, pins, etched trenches, or the like. In certain embodiments, the solid support may be porous. In some embodiments, the solid support is glass. In some embodiments, the solid support is coated. In some embodiments, the solid support is uncoated. In some embodiments, the coating adheres amines. In some embodiments, the coating adheres lysine residues. In some embodiments, the coating adheres amino termini of proteins. In some embodiments, the coating adheres 6-His sequences. In some embodiments, the coating adheres biotin residues.

Support materials useful in embodiments of the present invention include, for example, silicon, bio-compatible polymers such as, for example poly(methyl methacrylate) (PMMA) and polydimethylsiloxane (PDMS), glass, SiO2 (such as, for example, a thermal oxide silicon wafer such as that used by the semiconductor industry), quartz, silicon nitride, functionalized glass, gold, platinum, and aluminum. Functionalized surfaces include for example, amino-functionalized glass, carboxy functionalized glass, and hydroxy functionalized glass. Additionally, a support may optionally be coated with one or more layers to provide a surface for molecular attachment or functionalization, increased or decreased reactivity, binding detection, or other specialized application. Support materials and or layer(s) may be porous or non-porous. For example, a support may be comprised of porous silicon. Additionally, the support may be a silicon wafer or chip such as those used in the semiconductor device fabrication industry. In the case of a wafer or chip, a plurality of arrays may be synthesized on the wafer. A person skilled in the art would know how to select an appropriate support material.

In some embodiments, the plurality of probes is immobilized on the array. In some embodiments, the plurality of probes is linked to the array. In some embodiments, the immobilization is via linkage. In some embodiments, the plurality of probes is directly linked to the array. In some embodiments, the plurality of probes is indirectly linked to the array. In some embodiments, the linking is via a linker. In some embodiments, the linker is an amino acid linker. In some embodiments, the linker is at least one lysine residue. In some embodiments, the linker is a plurality of lysine residues. In some embodiments, the linker is two lysine residues. In some embodiments, the linker is KK. In some embodiments, the linker is at least one histidine residue. In some embodiments, the linker is a plurality of histidine residues. In some embodiments, the linker is six histidine residues. In some embodiments, the linker is HHHHHH (SEQ ID NO: 214). In some embodiments, the linker is a biotin residue. In some embodiments, the linker is an N-terminal linker. In some embodiments, the linker is a C-terminal linker. In some embodiments, the linker is an N-terminal or C-terminal linker.

The peptides, proteins and viruses present on the array may be linked covalently or non-covalently to the array and can be attached to the array support (e.g., silicon or other relatively flat material) by cleavable linkers. A linker molecule can be a molecule inserted between the support and peptide that is being synthesized, and a linker molecule may not necessarily convey functionality to the resulting peptide, such as molecular recognition functionality, but instead elongates the distance between the support surface and the peptide functionality to enhance the exposure of the peptide functionality on the surface of the support. Preferably a linker should be about 4 to about 120 atoms long to provide exposure. In some embodiments, a linker is at least 40 atoms long. In some embodiments, a linker is at least 48 atoms long. In some embodiments, a linker is between 40 and 150 atoms long. In some embodiments, a linker is about 48 atoms long. In some embodiments, a linker is about 120 atoms long. The linker molecules may be, for example, aryl acetylene, ethylene glycol oligomers containing 2-10 monomer units (PEGs), diamines, diacids, biotin, amino acids, among others, and combinations thereof. A person skilled in the art would know how to design appropriate linkers. In some embodiments, a probe is immobilized on the array but not linked. In some embodiments, a virus or protein is immobilized but not linked. In some embodiments, a link is reversible. In some embodiments, linking is printing the probe on the array. In some embodiments, a peptide is printed. In some embodiments, a recombinant protein is printed.

In some embodiments, each probe is located at a discrete location on a support. In some embodiments, each probe is located at a discrete location on an array. In some embodiments, each probe is immobilized at a discrete location. In some embodiments, each probe is distinctly immobilized. It will be understood by a skilled artisan that each probe must be able to be uniquely detected such that upon reading/scanning the array, the precise probe bound by an antibody can be determined. In some embodiments, each probe is immobilized on a separate support. In some embodiments, each probe is immobilized in a separate region of a support or array. In some embodiments, each probe is located or immobilized such that they can be uniquely measured or detected. In some embodiments, each probe is located or immobilized such that an antibody binding to the probe can be uniquely measured or detected.

In some embodiments, the plurality of probes comprises at least one probe from a first flavivirus. In some embodiments, the plurality of probes comprises at least one probe from a second flavivirus. In some embodiments, the first and second flavivirus are different flaviviruses. In some embodiments, the flavivirus is selected from the group consisting of: Zika virus, Dengue virus type 1, Dengue virus type 2, Dengue virus type 3, Dengue virus type 4, West Nile virus, Japanese encephalitis virus, Tick-borne encephalitis virus, Louping ill virus, Omsk hemorrhagic fever virus, Powassan virus, Apoi virus, Yokose virus, Yellow fever virus, Rocio virus, Ilheus Virus, Bagaza virus, St. Louis encephalitis virus, Murray Valley encephalitis virus, Alfuy Virus and Usutu virus. In some embodiments, the flavivirus is selected from the group consisting of: Zika virus, Dengue virus type 1, Dengue virus type 2, Dengue virus type 3, Dengue virus type 4, West Nile virus, Japanese encephalitis virus, and Tick-borne encephalitis virus. In some embodiments, the flaviviruses are Zika virus and Tick-borne encephalitis virus. In some embodiments, the flaviviruses are selected from Dengue virus type 1, Dengue virus type 2, Dengue virus type 3, and Dengue virus type 4.

As used herein the term “probe” refers to a part from a virus or a whole virus that contains at least one epitope that can be bound by an antibody. In some embodiments, a probe is a whole virus. In some embodiments, the probe is a virus-like particle (VLP). In some embodiments, a probe is a lysed virus. In some embodiments, a probe is a fraction from a lysed virus. In some embodiments, a probe is a whole protein. In some embodiments, the protein is a recombinant protein. In some embodiments, a probe is a portion of a protein. In some embodiments, the portion comprises a functional domain. In some embodiments, the protein is a peptide. In some embodiments, the probe comprises amino acids. In some embodiments, the probe comprises viral protein. In some embodiments, the probe comprises a viral epitope. In some embodiments, the epitope is an immunological epitope. In some embodiments, the probe is selected from a whole virus, a lysed virus, a VLP, a whole recombinant protein and a peptide.

As used herein, the terms “peptide”, and “ polypeptide ” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid. In some embodiments, the probe comprises a peptide. It will be understood that even a full virus will inherently comprise a peptide and it will comprise an amino acid. Similarly, a VLP and a recombinant protein must comprise a peptide.

In some embodiments, the peptide is a purified peptide. In some embodiments, the peptide is an isolated peptide. In some embodiments, the peptide is a recombinant peptide. In some embodiments, the peptide is a synthetic peptide. As used herein, the term “isolated peptide” refers to a peptide that is essentially free from contaminating cellular components, such as separate carbohydrate, lipid, or other proteinaceous impurities associated with the peptide in nature. In some embodiments, the peptide comprises post-translational modification. In some embodiments, an isolated peptide comprises post-translational modification. In some embodiments, the post-translational modification is glycosylation. Typically, a preparation of isolated peptide contains the peptide in a highly purified form, i.e., at least about 80% pure, at least about 90% pure, at least about 95% pure, greater than 95% pure, or greater than 99% pure. In some embodiments, a synthetic peptide is at least 99% pure. In some embodiments, a synthetic peptide is 100% pure.

In some embodiments, the peptide is a fragment of a flavivirus protein. In some embodiments, the peptide is a protein fragment that retains an immunogenic epitope. In some embodiments, the peptide is a linear peptide. In some embodiments, the peptide is a conformational peptide. In some embodiments, the peptide contains three-dimensional structure. In some embodiments, the peptide comprises a fusion loop of a flavivirus protein. In some embodiments, the peptide comprises a fusion loop of a flavivirus envelope protein. In some embodiments, the functional domain is a fusion loop. In some embodiments, a cysteine residue of a peptide has been mutated to another amino acid. Removal of cysteines removes disulfide bridges that may change the conformation of the peptide or render it non-linear. In some embodiments, the cysteine is mutated to a non-charged amino acid. In some embodiments, the cysteine is mutated to a non-charged amino acid. In some embodiments, the cysteine is mutated to a non-polar amino acid. In some embodiments, the cysteine is mutated to a methionine. In some embodiments, all cysteines are mutated. In some embodiments, the flavivirus protein is a surface protein.

In some embodiments, the peptide comprises a domain from a flavivirus protein. In some embodiments, the peptide comprises a functional domain from a flavivirus protein. In some embodiments, the peptide comprises a motif from a flavivirus protein. In some embodiments, the peptide comprises sufficient amino acids to retain a secondary structure found in an intact protein. In some embodiments, the secondary structure is a three-dimensional structure. In some embodiments, the functional domain is the fusion loop. In some embodiments, the peptide comprises a functional fragment of the protein. In some embodiments, the probe is functional. In some embodiments, the probe comprises a 3D functional epitope. In some embodiments, the probe comprises a conformational epitope. In some embodiments, the probe comprises a linear epitope.

In some embodiments, the probe is a full protein. In some embodiments, the protein is a recombinant protein. In some embodiments, the protein is an isolated protein. In some embodiments, the protein comprises a post-translational modification. In some embodiments, the post-translational modification is glycosylation. In some embodiments, the probe is a full virus. In some embodiments, the probe is a lysed virus. In some embodiments, the virus is an inactivated virus. It will be understood by a skilled artisan that full proteins and full viruses are likely to be properly folded and thus provide 3D, conformational epitopes, while peptide may or may not have conformational epitope and not just linear epitopes.

In some embodiments, the fusion loop comprises the full fusion loop domain. In some embodiments, the full fusion loop domain of ZIKA comprises SEQ ID NO: 3. In some embodiments, the full fusion loop domain of ZIKA consists of SEQ ID NO: 3. In some embodiments, a probe for adhering the fusion loop of ZIKA to the array comprises a KK linker. In some embodiments, a probe for adhering the fusion loop of ZIKA to the array comprises an HHEHHE (SEQ ID NO: 214) linker. In some embodiments, a fragment of the ZIKA fusion loop comprises a sequence selected from SEQ ID NO: 5, 6, 7, 8, 9, 34, 35, 36, 37, 38, 39 and 40. In some embodiments, a fragment of the ZIKA fusion loop consists of a sequence selected from SEQ ID NO: 5, 6, 7, 8, 9, 34, 35, 36, 37, 38, 39 and 40. In some embodiments, a probe comprises SEQ ID NO: 3. In some embodiments, a probe consists of SEQ ID NO: 3. In some embodiments, a probe comprises SEQ ID NO: 35. In some embodiments, a probe consists of SEQ ID NO: 35. In some embodiments, a probe for adhering the fragment of the fusion loop of ZIKA to the array comprises SEQ ID NO: 35. In some embodiments, a probe for adhering the fragment of the fusion loop of ZIKA to the array comprises SEQ ID NO: 39. In some embodiments, a probe for adhering the fragment of the fusion loop of ZIKA to the array consist of SEQ ID NO: 35. In some embodiments, a probe for adhering the fragment of the fusion loop of ZIKA to the array consists of SEQ ID NO: 39. In some embodiments, a probe for adhering the fusion loop of ZIKA to the array comprises SEQ ID NO: 3. In some embodiments, a probe for adhering the fusion loop of ZIKA to the array consists of SEQ ID NO: 3.

In some embodiments, a peptide comprises at least 5, 7, 10, 12, 14, 15, 16, 18, 20 or 25 amino acids. Each possibility represents a separate embodiment of the invention. In some embodiments, a peptide comprises at least 10 amino acids. In some embodiments, a peptide comprises at least 14 amino acids. In some embodiments, a peptide comprises at least 20 amino acids. In some embodiments, a peptide is not a complete protein. In some embodiments, a peptide comprises between 5 and 200, 5 and 150, 5 and 100, 5 and 90, 5 and 90, 5 and 70, 5 and 60, 5 and 58, 5 and 50, 10 and 200, 10 and 150, 10 and 100, 10 and 90, 10 and 90, 10 and 70, 10 and 60, 10 and 58, 10 and 50, 12 and 200, 12 and 150, 12 and 100, 12 and 90, 12 and 90, 12 and 70, 12 and 60, 12 and 58, 12 and 50, 14 and 200, 14 and 150, 14 and 100, 14 and 90, 14 and 90, 14 and 70, 14 and 60, 14 and 58, 14 and 50, 15 and 200, 15 and 150, 15 and 100, 15 and 90, 15 and 90, 15 and 70, 15 and 60, 15 and 58, or 15 and 50 amino acids. Each possibility represents a separate embodiment of the invention. In some embodiments, a peptide comprises between 5 and 60 amino acids. In some embodiments, a peptide comprises between 10 and 60 amino acids. In some embodiments, a peptide comprises between 14 and 60. In some embodiments, a peptide comprises between 5 and 58 amino acids. In some embodiments, a peptide comprises between 10 and 58 amino acids. In some embodiments, a peptide comprises between 14 and 58. In some embodiments, a peptide comprises at most 50, 58, 60, 70, 80, 90, 100, 125, 150, 175, 200 or 250 amino acids. Each possibility represents a separate embodiment of the invention. In some embodiments, a peptide comprises at most 60 amino acids. In some embodiments, a peptide comprises at most 100 amino acids. In some embodiments, the amino acids are consecutive amino acids from a flavivirus protein.

In some embodiments, a peptide is a protein. In some embodiments, a peptide is a part of a protein. In some embodiments, a peptide comprises a functional domain of a protein. In some embodiments, a peptide is a complete protein. In some embodiments, a complete protein is a whole protein. In some embodiments, a complete protein comprises a signal peptide. In some embodiments, a complete protein lacks a signal peptide. In some embodiments, the protein is a recombinant protein. In some embodiments, the probe is a complete protein. Recombinant proteins can be produced by any method known in the art, or can be purchased for commercial supplies, such as for example Sino Biological and The Native Antigen Company.

In some embodiments, the plurality of probes comprises a probe comprising an amino acid sequence of a flavivirus protein. In some embodiments, the plurality of probes comprises a probe consisting of an amino acid sequence of a flavivirus protein. In some embodiments, the plurality of probes comprises a protein probe from a first flavivirus. In some embodiments, the plurality of probes comprises a protein probe from a second flavivirus. In some embodiments, the protein is a surface protein. In some embodiments, the protein is a flavivirus envelope protein. In some embodiments, the envelope protein is an envelope glycoprotein. In some embodiments, the protein is a flavivirus cytoplasmic protein. In some embodiments, the cytoplasmic protein is a NS1 protein. In some embodiments, the protein is a recombinant protein. In some embodiments, the protein is a secreted protein. In some embodiments, the secreted protein is a NS1 protein. It will be understood by a skilled artisan that by using a protein, secondary structures and intramolecular bonds and interactions will be preserved. In some embodiments, a probe comprises a whole flavivirus protein. In some embodiments, a probe consists of a whole flavivirus protein. In some embodiments, the protein is selected from an envelope protein and an NS1 protein. In some embodiments, the plurality of probes comprises a first probe that consists of a whole protein and a second probe that consists of a whole protein.

In some embodiments, the plurality of probes comprises at least 1, 2, 3, 4, 5, 6, 7, 8, 9 or 10 peptides from a flavivirus. Each possibility represents a separate embodiment of the invention. In some embodiments, the plurality of probes comprises at least 2 peptides from a flavivirus. In some embodiments, the plurality of probes comprises 2 peptides from a flavivirus. In some embodiments, the plurality of probes comprises probes from at least 2 different flavivirus proteins. In some embodiments, the two proteins are an envelope protein and NS1. In some embodiments, the plurality of probes comprises at least 1, 2, 3, 4, 5, 6, 7, 8, 9 or 10 peptides from each flavivirus. Each possibility represents a separate embodiment of the invention. In some embodiments, the plurality of probes comprises at least 2 peptides from each flavivirus. In some embodiments, the plurality of probes comprises 2 peptides from each flavivirus. In some embodiments, at least 2 is 2. In some embodiments, the plurality of probes comprises at least a peptide from an envelope protein from each flavivirus. In some embodiments, the plurality of probes comprises at least a peptide from NS1 from each flavivirus. In some embodiments, the plurality of probes comprises at least the envelope protein from each flavivirus. In some embodiments, the plurality of probes comprises at least the NS1 protein from each flavivirus. In some embodiments, the peptide is the whole protein.

In some embodiments, an array comprises probes to at least 2 flaviviruses. In some embodiments, an array comprises probes to at least 3 flaviviruses. In some embodiments, an array comprises at least 10, 20, 30, 40, 50, 60, 70, 80, 90, 100, 150, 200, 250, 300, 350, 400, 450, 500, 550 or 600 probes. Each possibility represents a separate embodiment of the invention. In some embodiments, an array comprises at least 10 probes. In some embodiments, an array comprises at least 50 probes. In some embodiments, an array comprises at least 90 probes. In some embodiments, an array comprises at most 250, 300, 400, 500, 600, 700, 750, 800, 900 or 1000 probes. Each possibility represents a separate embodiment of the invention. In some embodiments, an array comprises at most 600 probes. In some embodiments, an array comprises 2-800, 10-800, 50-800, 90-800, 100-800, 200-800, 2-700, 10-700, 50-700, 90-700, 100-700, 200-70, 2-650, 10-650, 50-650, 90-650, 100-650, 200-650, 2-600, 10-600, 50-600, 90-600, 100-600, 200-600, 2-550, 10-550, 50-550, 90-550, 100-550, 200-550, 2-500, 10-500, 50-500, 90-500, 100-500, 200-500, 2-400, 10-400, 50-400, 90-400, 100-400, 200-400, 2-300, 10-300, 50-300, 90-300, 100-300, 200-300, 2-250, 10-250, 50-250, 90-250, 100-250, or 200-250. Each possibility represents a separate embodiment of the invention. In some embodiments, an array comprises 90-600 probes. In some embodiments, an array comprises 90-250 probes.

In some embodiments, the flavivirus is Zika virus (ZIKV). In some embodiments, the flavivirus is Dengue virus (DENG). In some embodiments, the Dengue virus is Dengue virus type 1 (DENG1). In some embodiments, the Dengue virus is Dengue virus type 2 (DENG2). In some embodiments, the Dengue virus is Dengue virus type 3 (DENG3). In some embodiments, the Dengue virus is Dengue virus type 4 (DENG4). In some embodiments, the flavivirus is West Nile virus (WNV). In some embodiments, the flavivirus is Japanese encephalitis virus (JEV). In some embodiments, the flavivirus is Tick-borne encephalitis virus (TBE). In some embodiments, the flavivirus is Louping ill virus. In some embodiments, the flavivirus is Omsk hemorrhagic fever virus. In some embodiments, the flavivirus is Powassan virus. In some embodiments, the flavivirus is Apoi virus. In some embodiments, the flavivirus is Yokose virus. In some embodiments, the flavivirus is Yellow fever virus (YFV). In some embodiments, the flavivirus is Rocio virus. In some embodiments, the flavivirus is Ilheus Virus. In some embodiments, the flavivirus is Bagaza virus. In some embodiments, the flavivirus is St. Louis encephalitis virus. In some embodiments, the flavivirus is Murray Valley encephalitis virus. In some embodiments, the flavivirus is Alfuy Virus. In some embodiments, the flavivirus is Usutu virus. In some embodiments, the virus is selected from Zika virus and Tick-borne encephalitis. In some embodiments, the virus is selected from Dengue virus type 1, Dengue virus type 2, Dengue virus type 3, and Dengue virus type 4.

In some embodiments, the plurality of probes comprises the same peptide from different flaviviruses. In some embodiments, the plurality of probes comprises the same whole protein from different flaviviruses. In some embodiments, the peptides from different flaviviruses are the same region of a peptide or protein but comprise different amino acid sequences. In some embodiments, the peptides are from the same region of the same protein from different flaviviruses. It will be understood that in different flaviviruses there will be mutations and other alterations in a given amino acids sequence and yet due to evolutionary conservation the proteins or peptides can be aligned, and common regions determined. Thus, the same peptide from one flavivirus to another, may be recognizable as the same peptide even though the sequence may be altered. Similarly, a given protein may be recognized as the same protein even if mutations have been generated in the amino acid sequence.

In some embodiments, the plurality of probes further comprises a flavivirus virus. In some embodiments, the flavivirus is an inactivated virus. As used herein, the term “inactivated virus” refers to a virulent virus that has been made non-infectious. In some embodiments, an inactivated virus is a killed virus. In some embodiments, an inactivated virus is a virus comprising a mutation that reduces virulence. In some embodiments, an inactivated virus is a virulent virus some of whose proteins have been transferred to a backbone of a less virulent or non-virulent virus. For example, the surface proteins from Dengue virus can be transplanted to a YFV virus backbone allowing for display of Dengue epitopes, but without the danger of active Dengue virus. In some embodiments, the flavivirus is a lysed virus. In some embodiments, the virus is a virion. In some embodiments, the lysed virus is a lysed cell infected by the virus. In some embodiments, the lysed virus is media from infected cells containing virus. In some embodiments, the virus is a virus-like particle. In some embodiments, the plurality of probes comprises a virus-like particle (VLP). In some embodiments, the VLP is a flavivirus VLP. As used herein, the term “virus-like particle” refers to a multiprotein structure that mimics the organization and conformation of an authentic native virus but lacks the viral genome. In some embodiments, the plurality of probes comprises a lysate from a cell infected by an flavivirus. In some embodiments, the lysate is mixed with spotting buffer before immobilization on the array or support. It will be appreciated by a skilled artisan that by using whole virus, VLPs or cell lysate viral epitopes will be provided in their natural confirmation. In some embodiments, the plurality of probes comprises at least two probes that are whole virus probes. In some embodiments, the plurality of probes comprises at least two VLP probes. In some embodiments, the plurality of probes comprises at least two lysed virus probes. In some embodiments, the plurality of probes comprises at least two virus probes that are different flaviviruses.

In some embodiments, the probes are present on the array or support at a concentration sufficient for antibody binding. In some embodiments, the probes are present on the array or support at a concentration sufficient for detectable antibody binding. In some embodiments, the concentration is at least 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 ng per spot of probe. Each possibility represents a separate embodiment of the invention. In some embodiments, the concentration is at least 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 ng/ml. Each possibility represents a separate embodiment of the invention. In some embodiments, the concentration is at most 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 ng per spot of probe. Each possibility represents a separate embodiment of the invention. In some embodiments, the concentration is at most 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 ng/ml. Each possibility represents a separate embodiment of the invention. In some embodiments, the concentration of protein is 8-32 ug/ml. In some embodiments, the concentration of peptide is -1 mg/ml. In some embodiments, the volume of the spot is at least 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 nL. Each possibility represents a separate embodiment of the invention. In some embodiments, the volume of the spot or probe is at most 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 nL. Each possibility represents a separate embodiment of the invention. In some embodiments, the volume of the spot or probe is ˜370 pL. In some embodiments, the spotted mass of whole recombinant proteins is between 3-20 picograms. In some embodiments, the spotted mass of peptides is between 300-400 picograms. It will be understood that shorter peptides will tend to have a lower mass and longer peptide a larger mass. In some embodiments, the spotted mass of peptide is ˜370 picograms.

In some embodiments, the plurality of probes is selected form the probes provided in Table 1, Table 2 or both. In some embodiments, the plurality of probes is selected form the probes provided in Table 1. In some embodiments, the plurality of probes is selected form the probes provided in Table 2. In some embodiments, the plurality of probes is selected form the probes provided in Table 1 and Table 2. In some embodiments, the plurality of probes comprises the probes provided in Table 1. In some embodiments, the plurality of probes consists of the probes provided in Table 1. In some embodiments, the plurality of probes comprises the probes provided in Table 2. In some embodiments, the plurality of probes consists of the probes provided in Table 2. In some embodiments, the plurality of probes comprises the probes provided in Table 1 and Table 2. In some embodiments, the plurality of probes consists of the probes provided in Table 1 and Table 2. In some embodiments, the plurality of probes comprises the probes provided in Table 1, Table 2 or both. In some embodiments, the plurality of probes consists of the probes provided in Table 1, Table 2 or both. In some embodiments, the plurality of probes comprises at least 10 probes selected from Table 1 and Table 2. In some embodiments, the plurality of probes comprises at least 5 probes from Table 1 and at least 5 probes from Table 2. In some embodiments, the plurality of probes comprises at least 20 probes selected from Table 1 and Table 2. In some embodiments, the plurality of probes comprises at least 10 probes from Table 1 and at least 10 probes from Table 2. In some embodiments, the plurality of probes comprises at least 20 probes selected from Table 1. In some embodiments, the plurality of probes comprises at least 20 probes selected from Table 2. In some embodiments, the plurality of probes comprises at least 10 probes selected from Table 1. In some embodiments, the plurality of probes comprises at least 10 probes selected from Table 2. In some embodiments, the plurality of probes comprises at least 30 probes selected from Table 1 and Table 2. In some embodiments, the plurality of probes comprises at least 40 probes selected from Table 1 and Table 2. In some embodiments, the plurality of probes comprises at least 50 probes selected from Table 1 and Table 2. In some embodiments, the plurality of probes comprises at least 100 probes selected from Table 1 and Table 2. In some embodiments, the plurality of probes comprises all the probes from Table 1 and Table 2. In some embodiments, the plurality of probes comprises all the probes from Table 1. In some embodiments, the plurality of probes comprises all the probes from Table 2.

In some embodiments, the plurality of probes comprises peptides selected from Table 2. In some embodiments, the plurality of probes consists of peptides selected from Table 2. In some embodiments, the plurality of probes consists of the peptides of Table 2. In some embodiments, the plurality of probes comprises peptides, wherein the peptides consist of the peptides of Table 2. In some embodiments, the plurality of probes consists of peptides selected from Table 2.

In some embodiments, the plurality of probes comprises viruses, VLPs or both selected from Table 1. In some embodiments, the plurality of probes consists of virus, VLPs or both selected from Table 1. In some embodiments, the plurality of probes consists of the viruses, VLPs or both of Table 1. In some embodiments, the plurality of probes comprises viruses, wherein the viruses consist of the viruses, VLPs or both of Table 1. In some embodiments, the plurality of probes consists of viruses, VLPs or both selected from Table 1.

In some embodiments, the plurality of probes comprises proteins selected from Table 1. In some embodiments, the plurality of probes consists of proteins selected from Table 1. In some embodiments, the plurality of probes consists of the proteins of Table 1. In some embodiments, the plurality of probes comprises proteins, wherein the proteins consist of the proteins of Table 1. In some embodiments, the plurality of probes consists of proteins selected from Table 1. In some embodiments, the plurality of probes consists of probes selected from Table 1. In some embodiments, the plurality of probes consists of probes selected from Table 1 and Table 2.

In some embodiments, the support or array consists of the plurality of probes. In some embodiments, the only probes on the array/support are the plurality of probes. In some embodiments, the solid support or array further comprises control probes. In some embodiments, control probes are probes that are bound by known antibodies found in all subjects. In some embodiments, control probes are probes that bind known antibodies found in all subjects. In some embodiments, control probes comprise secondary antibodies to human antibodies. In some embodiments, the secondary antibodies are selected from anti-human IgG, anti-human IgA and anti-human IgM. In some embodiments, control probes are peptides or proteins used to generate a vaccine. In some embodiments, the plurality of probes comprises control probes. In some embodiments, the control probes are from a non-flavivirus virus. In some embodiments, the non-flavivirus is an alphavirus. In some embodiments, the alphavirus is selected from O′nyong-nyong virus and Chikungunya virus. In some embodiments, a control is cell lysate from a cell uninfected by a flavivirus.

Table 2 lists various peptides that may be used on an array of the invention. A 58 amino acid peptide which is the fusion loop of various flaviviruses is provided. Several shorter peptides which are various fragments of the fusion loop of these flaviviruses are also provided. Peptides covering the envelope protein are provide, as are peptides covering the NS1 protein. In some embodiments, the plurality of probes comprises peptides spanning at least 10, 20, 25, 30, 40, 50, 60, 70, 75, 80, 90, 95, 99 or 100% of a flavivirus protein. Each possibility represents a separate embodiment of the invention. In some embodiments, the plurality of probes comprises peptides covering 100% of a flavivirus protein. In some embodiments, the peptide probes are selected from SEQ ID NOs: 1-213. In some embodiments, the peptide probes comprise peptides selected from SEQ ID NOs: 1-213. In some embodiments, the peptide probes comprise at least 2 peptides selected from SEQ ID NOs: 1-213. In some embodiments, the peptide probes comprise at least 2, 3, 4, 5, 6, 7, 8, 9, 10, 15, 20, 25, 30, 35, 40, 45, 50, 60, 70, 80, 90, 100, 110, 120, 125, 130, 140, 150, 160, 170, 175, 180, 190, 195, 200, 210 or 213 peptides selected from SEQ ID NOs: 1-213. Each possibility represents a separate embodiment of the invention. In some embodiments, the peptide probes comprise at least 2 peptides selected from SEQ ID NOs: 1-213. In some embodiments, the peptide probes comprise at least 10 peptides selected from SEQ ID NOs: 1-213.

TABLE 1
Protein and virus probes on an exemplary array

| Antigen ID | Description |
|---|---|
| recp_DENG1_rEnv | recombinant protein (DENG1 envelope) |
| recp_DENG2_rEnv | recombinant protein (DENG2 envelope) |
| recp_DENG3_rEnv | recombinant protein (DENG3 envelope) |
| recp_DENG4_rEnv | recombinant protein (DENG4 envelope) |
| recp_DENG1_rNS1 | recombinant protein (Dengue 1 NS1) |
| recp_DENG2_rNS1 | recombinant protein (Dengue 2 NS1) |
| recp_DENG3_rNS1 | recombinant protein (Dengue 3 NS1) |
| recp_DENG4_rNS1 | recombinant protein (Dengue 4 NS1) |
| recp_JEV_rEnv | recombinant protein (JEV envelope) |
| recp_JEV_rNS1 | recombinant protein (JEV NS1) |
| recp_TBE_rEnv | recombinant protein (TBE envelope) |
| recp_TBE_rNS1 | recombinant protein (TBE NS1) |
| recp_Usutu_rNS1 | recombinant protein (Usutu NS1) |
| recp_WNV_rEnv | recombinant protein (WNV envelope) |
| recp_WNV_rNS1 | recombinant protein (WNV NS1) |
| recp_YFV_rEnv | recombinant protein (YFV envelope) |
| recp_YFV_rNS1 | recombinant protein (YFV NS1) |
| recp_ZIKV_rEnv | recombinant protein (ZIKV envelope) |
| recp_ZIKV_rNS1 | recombinant protein (ZIKV NS1) |
| vac_TBE | TBE vaccine |
| ZIKA VLP | Virus-like particle |
| Dengue1 VLP | Virus-like particle |
| Dengue2 VLP | Virus-like particle |
| Dengue3 VLP | Virus-like particle |
| Dengue4 VLP | Virus-like particle |
| Chikungunya VLP | Virus-like particle |
| O'NY (O'nyong'nyong virus) VLP | Virus-like particle |
| Japanese Encephalitis Virus VLP | Virus-like particle |
| virus_Zika_Ag | whole inactivated virus (MR766 Uganda) |
| virus_YFV_Ag | whole inactivated virus (vaccine strain) |
| virus_Deng1_Ag | whole inactivated virus: E protein of DENG type 1 |
| | expressed on the base of YFV vaccine strain |
| virus_Deng2_Ag | whole inactivated viruss: E protein of DENG type2 |
| | expressed on the base of YFV vaccine strain |
| virus_Deng3_Ag | whole inactivated viruss: E protein of DENG type 3 |
| | expressed on the base of YFV vaccine strain |
| virus_Deng4_Ag | whole inactivated viruss: E protein of DENG type 4 |
| | expressed on the base of YFV vaccine strain |
| O'NY (O'nyong'nyong virus) | whole inactivated virus (Uganda) |
| antigen | |
| virus_Zika_Ag | lysate of VERO cells expressing the virus |
| | (MR766 from Uganda) |
| virus_YFV_Ag | lysate of VERO cells expressing the virus |
| | (vaccine strain) |
| virus_Deng1_Ag | lysate of VERO cells expressing the virus |
| | (E protein of DENG type 1 expressed on the |
| | base of YFV vaccine strain) |
| virus_Deng2_Ag | lysate of VERO cells expressing the virus |
| | (E protein of DENG type 2 expressed on the |
| | base of YFV vaccine strain) |
| virus_Deng3_Ag | lysate of VERO cells expressing the virus |
| | (E protein of DENG type 3 expressed on the |
| | base of YFV vaccine strain) |
| virus_Deng4_Ag | lysate of VERO cells expressing the virus |
| | (E protein of DENG type 4 expressed on the |
| | base of YFV vaccine strain) |
| O'NY (O'nyong'nyong virus) | lysate of VERO cells expressing the virus |
| antigen | (Uganda) |
| VERO cells lysate | lysate of uninfected VERO cells |

TABLE 2
Peptide probes on an exemplary array

| | | SEQ |
|---|---|---|
| | | ID |
| Antigen ID | sequences of peptides | NO: |
| WNV_EQUA | ATVSDLSTKAACPAGGEAHNDKRADPAFVCR | 1 |
| | QGVVDRGRGNGCGRFGKGSIDTCAKFA | |
| TBE_Full | AKLSDTKVAARCPTMGPATLAEEHQSGTVCKR | 2 |
| | DQSDRGWGNHCGLFGKGSIVTCVKAS | |
| ZIKV_Ful | ASISDMASDSRCPTQGEAYLDKQSDTQYVCKR | 3 |
| | TLVDRGWGNGCGLFGKGSLVTCAKFA | |
| ZIKV_EQU | ASISDMASDSRCPAGGEAYLDKQSDTQYVCKR | 4 |
| | TLVDRGRGNGCGRFGKGSLVTCAKFA | |
| ZFP_14 | DRGWGNGCGLFGKG | 5 |
| ZFP_N+4 | RTLVDRGWGNGCGLFGKG | 6 |
| ZFP_C+4 | DRGWGNGCGLFGKGSLVT | 7 |
| ZFP_22 | RTLVDRGWGNGCGLFGKGSLVT | 8 |
| ZFP_32 | QYVCKRTLVDRGWGNGCGLFGKGSLVTCAKF | 9 |
| | A | |
| ZFPM_N9 | QYVCKRTLVDRGWGNGCGLFGKG | 10 |
| ZFPM_C9 | DRGWGNGCGLFGKGSLVTCAKFA | 11 |
| ZFPM_14 | DRGWGNGMGLFGKG | 12 |
| ZFPM_N4 | RTLVDRGWGNGMGLFGKG | 13 |
| ZFPM_C4 | DRGWGNGMGLFGKGSLVT | 14 |
| ZFPM_22 | RTLVDRGWGNGMGLFGKGSLVT | 15 |
| TFPM_14 | DRGWGNHMGLFGKG | 16 |
| TFPM_N4 | RDQSDRGWGNHMGLFGKG | 17 |
| TFPM_C4 | DRGWGNHMGLFGKGSIVT | 18 |
| TFPM_22 | RDQSDRGWGNHMGLFGKGSIVT | 19 |
| WFPM_14 | DRGWGNGMGLFGKG | 12 |
| WFPM_N4 | QGVVDRGWGNGMGLFGKG | 20 |
| WFPM_C4 | DRGWGNGMGLFGKGSIDT | 21 |
| WFPM_22 | QGVVDRGWGNGMGLFGKGSIDT | 22 |
| D2FVM_14 | DRGWGNGMGLFGKG | 12 |
| D2FVM_N4 | HSMVDRGWGNGMGLFGKG | 23 |
| D2FVM_C4 | DRGWGNGMGLFGKGGIVT | 24 |
| D2FVM_22 | HSMVDRGWGNGMGLFGKGGIVT | 25 |
| EQUAM_14 | DRGRGNGMGRFGKG | 26 |
| EQUAM_N4 | QGVVDRGRGNGMGRFGKG | 27 |
| EQUAM_C4 | DRGRGNGMGRFGKGSIDT | 28 |
| EQUAM_22 | QGVVDRGRGNGMGRFGKGSIDT | 29 |
| ZE_1 | MSGGTWVDVVLEHGGCVTVM | 30 |
| ZE_2 | LEHGGCVTVMAQDKPTVDIE | 31 |
| ZE_3 | AQDKPTVDIELVTTTVSNMA | 32 |
| ZE_4 | LVTTTVSNMAEVRSYCYEAS | 33 |
| ZE_5 | EVRSYCYEASISDMASDSRC | 34 |
| ZE_6 | ISDMASDSRCPTQGEAYLDK | 35 |
| ZE_7 | PTQGEAYLDKQSDTQYVCKR | 36 |
| ZE_8 | QSDTQYVCKRTLVDRGWGNG | 37 |
| ZE_9 | TLVDRGWGNGCGLFGKGSLV | 38 |
| ZE_10 | CGLFGKGSLVTCAKFACSKK | 39 |
| ZE_11 | TCAKFACSKKMTGKSIQPEN | 40 |
| ZE_12 | MTGKSIQPENLEYRIMLSVH | 41 |
| ZE_13 | LEYRIMLSVHGSQHSGMIVN | 42 |
| ZE_14 | GSQHSGMIVNDTGHETDENR | 43 |
| ZE_15 | DTGHETDENRAKVEITPNSP | 44 |
| ZE_16 | AKVEITPNSPRAEATLGGFG | 45 |
| ZE_17 | RAEATLGGFGSLGLDCEPRT | 46 |
| ZE_18 | SLGLDCEPRTGLDFSDLYYL | 47 |
| ZE_19 | GLDFSDLYYLTMNNKHWLVH | 48 |
| ZE_20 | TMNNKHWLVHKEWFHDIPLP | 49 |
| ZE_21 | EWFHDIPLPWHAGADTGTP | 50 |
| ZE_22 | WHAGADTGTPEALVE | 51 |
| ZE_23 | HWNNKEALVEFKDAHAKRQT | 52 |
| ZE_24 | FKDAHAKRQTVVVLGSQEGA | 53 |
| ZE_25 | VVVLGSQEGAVHTALAGALE | 54 |
| ZE_26 | VHTALAGALEAEMDGAKGRL | 55 |
| ZE_27 | AEMDGAKGRLSSGHLKCRLK | 56 |
| ZE_28 | SSGHLKCRLKMDKLRLKGVS | 57 |
| ZE_29 | MDKLRLKGVSYSLCTAAFTF | 58 |
| ZE_30 | YSLCTAAFTFTKIPAETLHG | 59 |
| ZE_31 | TKIPAETLHGTVTVEVQYAG | 60 |
| ZE_32 | TVTVEVQYAGTDGPCKVPAQ | 61 |
| ZE_33 | TDGPCKVPAQMAVDMQTLTP | 62 |
| ZE_34 | MAVDMQTLTPVGRLITANPV | 63 |
| ZE_35 | VGRLITANPVITESTENSKM | 64 |
| ZE_36 | ITESTENSKMMLELDPPFGD | 65 |
| ZE_37 | MLELDPPFGDSYIVIGVGEK | 66 |
| ZE_38 | SYIVIGVGEKKITHHWHRSG | 67 |
| ZE_39 | ITHHWHRSGSTIGKAFEAT | 68 |
| ZE_40 | STIGKAFEATVRGAKRMAVL | 69 |
| ZE_41 | VRGAKRMAVLGDTAWDFGSV | 70 |
| ZE_42 | GDTAWDFGSVGGALNSLGKG | 71 |
| ZE_43 | GGALNSLGKGIHQIFGAAFK | 72 |
| ZE_44 | IHQIFGAAFKSLFGGMSWFS | 73 |
| ZE_45 | SLFGGMSWFSQILIGTLLMW | 74 |
| ZE_46 | QILIGTLLMWLGLNTKNGSI | 75 |
| ZE_47 | LGLNTKNGSISLMCLALGG | 76 |
| MR766_E_14 | GSQHSGMIGYETDEDRAKVE | 77 |
| MR766_E_15 | MIGYETDEDRAKVEVTPNSP | 78 |
| TBE_FE_E_8 | AARCPTMGPATLAEEHQSGT | 79 |
| TBE_FE_E_9 | TLAEEHQSGTVCKRDQSDRG | 80 |
| ZIKV_NS1_1 | DVGCSVDFSKKETRCGTGVF | 81 |
| ZIKV_NS1_2 | ETRCGTGVFIYNDVEAWRD | 82 |
| ZIKV_NS1_3 | IYNDVEAWRDRYKYHPDSPR | 83 |
| ZIKV_NS1_4 | RYKYHPDSPRRLAAAVKQAW | 84 |
| ZIKV_NS1_5 | RLAAAVKQAWEEGICGISSV | 85 |
| ZIKV_NS1_6 | EEGICGISSVSRMENIMWKS | 86 |
| ZIKV_NS1_7 | SRMENIMWKSVEGELNAILE | 87 |
| ZIKV_NS1_8 | VEGELNAILEENGVQLTVVV | 88 |
| ZIKV_NS1_9 | ENGVQLTVVVGSVKNPMWRG | 89 |
| ZIKV_NS1_10 | GSVKNPMWRGPQRLPVPVNE | 90 |
| ZIKV_NS1_11 | PQRLPVPVNELPHGWKAWGK | 91 |
| ZIKV_NS1_12 | LPHGWKAWGKSYFVRAAKTN | 92 |
| ZIKV_NS1_13 | SYFVRAAKTNNSFVVDGDTL | 93 |
| ZIKV_NS1_14 | NSFVVDGDTLKECPLEHRAW | 94 |
| ZIKV_NS1_15 | ECPLEHRAWNSFLVEDHGF | 95 |
| ZIKV_NS1_16 | NSFLVEDHGFGVFHTSVWLK | 96 |
| ZIKV_NS1_17 | GVFHTSVWLKVREDYSLECD | 97 |
| ZIKV_NS1_18 | VREDYSLECDPAVIGTAVKG | 98 |
| ZIKV_NS1_19 | PAVIGTAVKGREAAHSDLGY | 99 |
| ZIKV_NS1_20 | REAAHSDLGYWIESEKNDTW | 100 |
| ZIKV_NS1_21 | WIESEKNDTWRLKRAHLIEM | 101 |
| ZIKV_NS1_22 | RLKRAHLIEMKTCEWPKSHT | 102 |
| ZIKV_NS1_23 | TCEWPKSHTLWTDGVEESD | 103 |
| ZIKV_NS1_24 | LWTDGVEESDLIIPKSLAGP | 104 |
| ZIKV_NS1_25 | LIIPKSLAGPLSHHNTREGY | 105 |
| ZIKV_NS1_26 | LSHHNTREGYRTQVKGPWHS | 106 |
| ZIKV_NS1_27 | RTQVKGPWHSEELEIRFEEC | 107 |
| ZIKV_NS1_28 | EELEIRFEECPGTKVYVEET | 108 |
| ZIKV_NS1_29 | PGTKVYVEETCGTRGPSLRS | 109 |
| ZIKV_NS1_30 | CGTRGPSLRSTTASGRVIEE | 110 |
| ZIKV_NS1_31 | TTASGRVIEEWCCRECT1VIPP | 111 |
| ZIKV_NS1_32 | WCCRECT1VIPPLSFRAKDGCW | 112 |
| ZIKV_NS1_33 | L SFRAKDGCWYGMEIRPRKE | 113 |
| ZIKV_NS1_34 | YGMEIRPRKEPESNLVRSMV | 114 |
| ZIKV_NS1_35 | PESNLVRSMVTAGS | 115 |
| TBE_E_1 | SRCTHLENRDFVTGTQGTTR | 116 |
| TBE_E_2 | FVTGTQGTTRVTLVLELGGC | 117 |
| TBE_E_3 | VTLVLELGGCVTITAEGKPS | 118 |
| TBE_E_4 | VTITAEGKPSMDVWLDAIYQ | 119 |
| TBE_E_5 | MDVWLDAIYQENPAKTREYC | 120 |
| TBE_E_6 | ENPAKTREYCLHAKLSDTKV | 121 |
| TBE_E_7 | LHAKLSDTKVAARCPTMGPA | 122 |
| TBE_E_8 | AARCPTMGPATLAEEHQGGT | 123 |
| TBE_E_9 | TLAEEHQGGTVCKRDQSDRG | 124 |
| TBE_E_10 | VCKRDQSDRGWGNHCGLFGK | 125 |
| TBE_E_11 | WGNHCGLFGKGSIVACVKAA | 126 |
| TBE_E_12 | GSIVACVKAACEAKKKATGH | 127 |
| TBE_E_13 | CEAKKKATGHVYDANKIVYT | 128 |
| TBE_E_14 | VYDANKIVYTVKVEPHTGDY | 129 |
| TBE_E_15 | VKVEPHTGDYVAANETHSGR | 130 |
| TBE_E_16 | VAANETHSGRKTASFTISSE | 131 |
| TBE_E_17 | TASFTISSEKTILTMGEYG | 132 |
| TBE_E_18 | TILTMGEYGDVSLLCRVAS | 133 |
| TBE_E_19 | DVSLLCRVASGVDLAQTVIL | 134 |
| TBE_E_20 | GVDLAQTVILELDKTVEHLP | 135 |
| TBE_E_21 | ELDKTVEHLPTAWQVHRDWF | 136 |
| TBE_E_22 | TAWQVHRDWFNDLALPWKHE | 137 |
| TBE_E_23 | NDLALPWKHEGAQNWNNAER | 138 |
| TBE_E_24 | GAQNWNNAERLVEFGAPHAV | 139 |
| TBE_E_25 | LVEFGAPHAVKMDVYNLGDQ | 140 |
| TBE_E_26 | KMDVYNLGDQTGVLLKALAG | 141 |
| TBE_E_27 | TGVLLKALAGVPVAHIEGTK | 142 |
| TBE_E_28 | VPVAHIEGTKYHLKSGHVTC | 143 |
| TBE_E_29 | YHLKSGHVTCEVGLEKLKMK | 144 |
| TBE_E_30 | EVGLEKLKMKGLTYTMCDKT | 145 |
| TBE_E_31 | GLTYTMCDKTKFTWKRAPTD | 146 |
| TBE_E_32 | KFTWKRAPTDSGHDTVVMEV | 147 |
| TBE_E_33 | SGHDTVVMEVTFSGTKPCRI | 148 |
| TBE_E_34 | TFSGTKPCRIPVRAVAHGSP | 149 |
| TBE_E_35 | PVRAVAHGSPDVNVAMLITP | 150 |
| TBE_E_36 | DVNVAMLITPNPTIENNGGG | 151 |
| TBE_E_37 | NPTIENNGGGFIEMQLPPGD | 152 |
| TBE_E_38 | FIEMQLPPGDNIIYVGELSH | 153 |
| TBE_E_39 | NIIYVGELSHQWFQKGSSIG | 154 |
| TBE_E_40 | QWFQKGSSIGRVFQKTKKGI | 155 |
| TBE_E_41 | RVFQKTKKGIERLTVIGEHA | 156 |
| TBE_E_42 | ERLTVIGEHAWDFGSAGGFL | 157 |
| TBE_E_43 | WDEGSAGGELSSIGKAVHTV | 158 |
| TBE_E_44 | SSIGKAVHTVLGGAFNSIFG | 159 |
| TBE_E_45 | LGGAFNSIFGGVGFLPKLLL | 160 |
| TBE_E_46 | GVGFLPKLLLGVALAWLGLN | 161 |
| TBE_E_47 | GVALAWLGLNMRNPTMSMSF | 162 |
| TBE_E_48 | MRNPTMSMSFLLAGGLVLAM | 163 |
| TBE_E_49 | LLAGGLVLAMTLGVGA | 164 |
| TBE_NS1_1 | DVGCAVDTERMELRCGEGLV | 165 |
| TBE_NS1_2 | MELRCGEGLVVWREVSEWYD | 166 |
| TBE_NS1_3 | VWREVSEWYDNYAYYPETPG | 167 |
| TBE_NS1_4 | NYAYYPETPGALASAIKETF | 168 |
| TBE_NS1_5 | ALASAIKETFEEGSCGVVPQ | 169 |
| TBE_NS1_6 | EEGSCGVVPQNRLEMAMWRS | 170 |
| TBE_NS1_7 | NRLEMAMWRSSVTELNLALA | 171 |
| TBE_NS1_8 | SVTELNLALAEGEANLTVVV | 172 |
| TBE_NS1_9 | EGEANLTVVVDKFDPTDYRG | 173 |
| TBE_NS1_10 | DKFDPTDYRGGVPGLLKKGK | 174 |
| TBE_NS1_11 | GVPGLLKKGKDIKVSWKSWG | 175 |
| TBE_NS1_12 | DIKVSWKSWGHSMIWSIPEA | 176 |
| TBE_NS1_13 | HSMIWSIPEAPRRFMVGTEG | 177 |
| TBE_NS1_14 | PRRFMVGTEGQSECPLERRK | 178 |
| TBE_NS1_15 | QSECPLERRKTGVFTVAEFG | 179 |
| TBE_NS1_16 | TGVFTVAEFGVGLRTKVFLD | 180 |
| TBE_NS1_17 | VGLRTKVFLDFRQEPTHECD | 181 |
| TBE_NS1_18 | FRQEPTHECDTGVMGAAVKN | 182 |
| TBE_NS1_19 | TGVMGAAVKNGMAIHTDQSL | 183 |
| TBE_NS1_20 | GMAIHTDQSLWMRSMKNDTG | 184 |
| TBE_NS1_21 | WMRSMKNDTGTYIVELLVTD | 185 |
| TBE_NS1_22 | TYIVELLVTDLRNCSWPASH | 186 |
| TBE_NS1_23 | LRNCSWPASHTIDNADVVDS | 187 |
| TBE_NS1_24 | TIDNADVVDSELFLPASLAG | 188 |
| TBE_NS1_25 | ELFLPASLAGPRSWYNRIPG | 189 |
| TBE_NS1_26 | PRSWYNRIPGYSEQVKGPWK | 190 |
| TBE_NS1_27 | YSEQVKGPWKYTPIRVIREE | 191 |
| TBE_NS1_28 | YTPIRVIREECPGTTVTINA | 192 |
| TBE_NS1_29 | CPGTTVTINAKCDKRGASVR | 193 |
| TBE_NS1_30 | CDKRGASVRSTTESGKVIP | 194 |
| TBE_NS1_31 | STTESGKVIPEWCCRACT1VIP | 195 |
| TBE_NS1_32 | EWCCRACT1VIPPVTFRTGTDC | 196 |
| TBE_NS1_33 | PVTFRTGTDCWYAMEIRPVH | 197 |
| TBE_NS1_34 | WYAMEIRPVHDQGGLVRSMV | 198 |
| TBE_NS1_35 | DQGGLVRSMVVA | 199 |
| TFP_14 | DRGWGNHCGLFGKG | 200 |
| TFP_N4 | RDQSDRGWGNHCGLFGKG | 201 |
| TFP_C4 | DRGWGNHCGLFGKGSIVT | 202 |
| TFP_22 | RDQSDRGWGNHCGLFGKGSIVT | 203 |
| WFP_14 | DRGWGNGCGLFGKG | 5 |
| WFP_N4 | QGVVDRGWGNGCGLFGKG | 204 |
| WFP_C4 | DRGWGNGCGLFGKGSIDT | 205 |
| WFP_22 | QGVVDRGWGNGCGLFGKGSIDT | 206 |
| D2FV_14 | DRGWGNGCGLFGKG | 5 |
| D2FV_N4 | HSMVDRGWGNGCGLFGKG | 207 |
| D2FV_C4 | DRGWGNGCGLFGKGGIVT | 208 |
| D2FV_22 | HSMVDRGWGNGCGLFGKGGIVT | 209 |
| EQUA_14 | DRGRGNGCGRFGKG | 210 |
| EQUA_N4 | QGVVDRGRGNGCGRFGKG | 211 |
| EQUA_C4 | DRGRGNGCGRFGKGSIDT | 212 |
| EQUA_22 | QGVVDRGRGNGCGRFGKGSIDT | 213 |

According to another aspect, there is provided a kit comprising an array or support of the invention.

According to another aspect, there is provided a system comprising an array or support of the invention.

In some embodiments, the kit further comprises a detecting agent. In some embodiments, the kit further comprises at least one detecting agent. In some embodiments, the detecting agent is a labeled detecting agent. In some embodiments, the detecting agent is for detecting binding of an antibody to a probe of the array or support. In some embodiments, the detecting agent is for detecting antibodies. In some embodiments, the detecting agent is for detecting antibodies from a subject. In some embodiments, the detecting agent is for detecting human antibodies. In some embodiments, the detecting agent is for detecting IgG, IgA, IgM or a combination thereof. In some embodiments, the detecting agent is for detecting IgA. In some embodiments, the detecting agent is at least one labeled secondary antibody. In some embodiments, the secondary antibody is configured for detection of antibodies bound to the array or support. In some embodiments, the secondary antibody is an anti-human secondary antibody. Antibodies against any organism for which sample is to be tested can be included in the kit. In some embodiments, the secondary antibody is an anti-IgG antibody. In some embodiments, the secondary antibody is an anti-IgA antibody. In some embodiments, the secondary antibody is an anti-IgM antibody. In some embodiments, agents for detecting IgM, IgA and IgG comprise distinct labels.

In some embodiments, the kit comprises at least 1, 2, 3, 4, 5, 6, 7, 8, 9, or 10 secondary antibodies. Each possibility represents a separate embodiment of the invention. In some embodiments, the kit comprises at least 2 secondary antibodies. In some embodiments, the kit comprises an anti-IgG and an anti-IgA antibody. In some embodiments, each secondary antibody comprises a uniquely detectable label. As such, the binding of each secondary antibody can be measured separately, or simultaneously but distinctly identified.

In some embodiments, the label is a fluorescent label. In some embodiments, the label is a radioactive label. Detectable labels are well known in the art and any uniquely detectable label may be used.

In some embodiments, the system comprises a detector or sensor configured to detect binding of antibodies to probes of the array or support. In some embodiments, the detector or sensor is configured to detect labeled secondary antibodies. In some embodiments, the detector or support is configured to detect fluorescence. In some embodiments, the detector or sensor is configured to detect binding at specific locations on the array or support. In some embodiments, the detector or sense is configured to detect binding of antibodies to probes immobilized on the array or support.

In some embodiments, the array or support is for use in determining the presence of cross-reactive antibodies to a flavivirus in a sample. In some embodiments, the array or support is for use in assessing the risk of a subject developing antibody dependent enhancement (ADE) upon infection with a flavivirus. In some embodiments, the array or support is for use in predicting the future risk of ADE induction due to vaccination by a flavivirus vaccine. In some embodiments, the array or support is for use in determining suitability of a subject to receive a flavivirus vaccine. In some embodiments, the array or support is for use in predicting the effectiveness of a flavivirus vaccination in a given subject. In some embodiments, the array or support is for use in detecting previous flavivirus infection in a subject. In some embodiments, the array or support is for use in detecting vaccination in a subject. In some embodiments, the array or support is for use in determining the flavivirus that had previously infected a subject. In some embodiments, the array or support is for use in determining the flavivirus that a subject had previously been vaccinated against. In some embodiments, the array or support is for use in determining the potency of a flavivirus vaccine. In some embodiments, the array or support is for use in determining the efficacy of a flavivirus vaccine. It will be understood by a skilled artisan that any use for which the array or support can be used, so too a kit or system of the invention can also be used. m

According to another aspect, there is provided a method of determining the suitability of a subject in need thereof to receive a flavivirus vaccination, the method comprising providing a sample from the subject, contacting the sample to an array or support of the invention, detecting binding of an antibody from the sample to a discrete location on the array or support, and generating a flavivirus immune score from the detected binding, thereby determining the suitability of a subject to receive a flavivirus vaccination.

According to another aspect, there is provided a method of predicting effectiveness of a flavivirus vaccine in a subject in need thereof, the method comprising providing a sample from the subject, contacting the sample to an array or support of the invention, detecting binding of an antibody from the sample to a discrete location on the array or support, and generating a flavivirus immune score from the detected binding, thereby predicting effectiveness of a flavivirus vaccine in a subject.

According to another aspect, there is provided a method of predicting the effectiveness of a flavivirus vaccine, the method comprising: providing a solution comprising antibodies from immune cells contacted by the flavivirus vaccine; contacting the solution to an array or support of the invention; detecting binding of the antibodies to locations on the array or support, and generating a flavivirus immune score from the detected binding; thereby predicting the effectiveness of a flavivirus vaccine.

According to another aspect, there is provided a method of measuring cross-reactive antibodies in a subject in need thereof, the method comprising: providing a sample from the subject, contacting the sample to an array or support of the invention, detecting binding of an antibody from the sample to a discrete location on the array or support, thereby measuring cross-reactive antibodies.

According to another aspect, there is provided a method of asses a risk of a subject in need thereof, of developing ADE upon infection by a flavivirus, the method comprising: providing a sample from the subject, contacting the sample to an array or support of the invention, detecting binding of an antibody from the sample to a discrete location on the array or support, and generating a flavivirus immune score from the detected binding, thereby assessing the risk of a subject developing ADE upon infection by a flavivirus.

According to another aspect, there is provided a method of predicting a risk of future ADE induction due to vaccination by a flavivirus vaccine, the method comprising: providing a solution comprising antibodies from immune cells contacted by the flavivirus vaccine; contacting the solution to an array or support of the invention; detecting binding of the antibodies to locations on the array or support, and generating a flavivirus immune score from the detected binding; thereby predicting the risk of future ADE induction due to vaccination by the flavivirus vaccine.

In some embodiments, the method is an in vitro method. In some embodiments, the method is an ex vivo method. In some embodiments, the method is a diagnostic method. In some embodiments, the method further comprises administering the vaccine to a subject in need thereof. In some embodiments, the method further comprises administering a different vaccine than one that produces cross-reactive antibodies. In some embodiments, the method further comprises administering a prophylactic treatment.

In some embodiments, the subject is a human. In some embodiments, the subject is a lab animal. In some embodiments, the subject is a veterinary animal. In some embodiments, the subject is a wild animal. In some embodiments, the subject is a mammal. In some embodiments, the subject is avian. In some embodiments, the subject is at risk for contracting a flavivirus. In some embodiments, the subject has previously been vaccinated against a flavivirus. In some embodiments, the subject has previously been infected by a flavivirus. In some embodiments, the subject is naïve to a flavivirus. In some embodiments, the subject is an infant. In some embodiments, the subject is elderly. In some embodiments, the subject is a child. In some embodiments, the subject is an adult. In some embodiments, the subject is pregnant or at risk of becoming pregnant. In some embodiments, the subject is preparing to travel to a location where infection by a flavivirus is prevalent.

In some embodiments, the sample is a biological sample. In some embodiments, the sample is a bodily fluid. In some embodiments, the bodily fluid is selected from: blood, serum, gastric fluid, intestinal fluid, saliva, nasal swab, oral swab, tracheal swab, bile, breast milk, urine, interstitial fluid, and stool. In some embodiments, the bodily fluid is blood. In some embodiments, the bodily fluid is serum. In some embodiments, the bodily fluid is plasma. In some embodiments, the blood is peripheral blood. In some embodiments, the bodily fluid is selected from blood and serum. In some embodiments, the bodily fluid is selected from blood, plasma and serum. In some embodiments, the bodily fluid is saliva. In some embodiments, the bodily fluid is a nasal swab. In some embodiments, the bodily fluid is an oral swab. In some embodiments, the bodily fluid is selected from blood, saliva, nasal swab, oral swab and serum. In some embodiments, the bodily fluid is selected from blood, saliva and serum. In some embodiments, the sample is from the subject. In some embodiments, the sample is a sample comprising antibodies. In some embodiments, the sample comprises antibodies from the subject.

In some embodiments, the sample comprises at least 1, 2, 3, 4, 5, 6, 7, 8, 9, or 10 microliters of fluid. Each possibility represents a separate embodiment of the invention. In some embodiments, the sample comprises at most 10, 15, 20, 25, 30, 35, 40, 45, 50, 55, 60, 65, 70, 75, 80, 85, 90, 95, 100, 200, 400 or 500 microliters of fluid. Each possibility represents a separate embodiment of the invention. In some embodiments, the sample comprises sufficient liquid to cover the array or support. In some embodiments, the sample comprises sufficient liquid to cover the plurality of probes. In some embodiments, the sample is diluted in buffer. In some embodiments, the buffer is binding buffer.

In some embodiments, the contacting is in conditions sufficient for antibody binding to the probes. In some embodiments, the contacting is in conditions sufficient for antibody binding to the plurality of probes. Conditions for antibody binding will be known by one skilled in the art. Further, optimization of binding conditions can be determined by a skilled artisan. In some embodiments, the contacting produces an array or support with bound antibodies. In some embodiments, the contacting produces antibodies bound to the array or support.

In some embodiments, the method is a method of measuring cross-reactive antibodies. In some embodiments, the antibodies are cross-reactive to a flavivirus. In some embodiments, the measuring is detecting. In some embodiments, the detecting comprises detecting binding of an antibody to a probe. In some embodiments, the binding indicates the presence in the sample of an antibody to a probe located at the detected location. In some embodiments, the binding indicates the present in the sample of an antibody to a given peptide, protein, or virus. The identity of the virus, peptide or protein to which there are antibodies in the sample is determined by the known locations of each probe.

In some embodiments, the detecting comprises contacting the array or support with bound antibodies with a labeled detecting agent. In some embodiments, the detecting agent is a secondary antibody. In some embodiments, the detecting agent detects antibodies from the sample. In some embodiments, the detecting agent detects binding of antibodies to a target. In some embodiments, the detecting agent detects binding of antibody from the sample to a probe of the array or support.

In some embodiments, the method is a method of assessing a risk of the subject developing ADE. In some embodiments, the risk of developing ADE is upon infection by a flavivirus. In some embodiments, the infection by a flavivirus is the flavivirus for which the subject has cross-reactive antibodies. In some embodiments, the method further comprises generating a flavivirus immune score. In some embodiments, the immune score is generated from the detected binding. In some embodiments, the magnitude of the immune score is proportional to the risk of developing ADE.

In some embodiments, generating a flavivirus immune score comprises summing the magnitude of binding of antibodies to given probes. In some embodiments, given probes are all the probes for a specific flavivirus. In some embodiments, the flavivirus immune score is virus specific. In some embodiments, the flavivirus immune score is informative for a plurality of flaviviruses. In some embodiments, the flavivirus immune score is informative for all flaviviruses. In some embodiments, generating an flavivirus immune score comprises summing the magnitude of binding of antibodies to all probes. In some embodiments, generating a flavivirus immune score comprises performing an algorithm as provided herein below. In some embodiments, binding to specific probes is weighted and the sum of the magnitudes of binding comprises these weights. In some embodiments, binding to specific probes is weighted such that binding to certain probes has a greater impact on the immune score and binding to other probes has a lesser impact on immune score. For example, for three probes x, y, and z an immune score may be compiled by summing binding to x+binding to y+binding to z. Alternatively, x, y and z may be given weights a, b and c respectively and thus the immune score would be calculated by a*x+b*y+c*z. In some embodiments, a flavivirus immune score is generated only from IgA binding. In some embodiments, a flavivirus immune score is generated only from IgG binding. In some embodiments, a flavivirus immune score is generated from binding to peptides. In some embodiments, a flavivirus immune score is generated from binding to proteins. In some embodiments, a flavivirus immune score is generated from binding to virus. In some embodiments, a flavivirus immune score is generated from binding to VLPs.

In some embodiments, the immune score is proportional to the subject's suitability to receive a flavivirus vaccine. In some embodiments, the immune score is the magnitude of the immune score. In some embodiments, the immune score is the magnitude of binding. In some embodiments, the immune score is a numerical value. In some embodiments, immune score is proportional to the effectiveness of a flavivirus vaccine in the subject. In some embodiments, immune score is proportional to the effectiveness of a flavivirus vaccine on the subject. In some embodiments, effectiveness is predicted effectiveness. In some embodiments, a subject with low predicted effectiveness is not suitable to receive the vaccination. In some embodiments, a predicted effectiveness below a predestined threshold indicates the subject is not suitable to receive the vaccine.

In some embodiments, the immune score is proportional to the subject's risk of developing ADE. In some embodiments, the developing ADE is upon future infection by a flavivirus. In some embodiments, the immune score is proportional to the risk of a vaccine in causing future ADE to a subject that receives the vaccine. In some embodiments, future ADE is developing ADE. In some embodiments, the developing ADE is for a particular flavivirus. In some embodiments, the ADE is Zika ADE. In some embodiments, the ADE is Dengue ADE.

In some embodiments, a higher immune score indicates a greater suitability to receive a flavivirus vaccine. In some embodiments, a higher immune score indicates a lesser suitability to receive a flavivirus vaccine. In some embodiments, a higher immune score indicates a greater likelihood of effectiveness of a flavivirus vaccine. In some embodiments, a lower immune score indicates a greater likelihood of effectiveness of a flavivirus vaccine. In some embodiments, a higher immune score indicates a greater likelihood of effectiveness of a flavivirus vaccine in the subject. In some embodiments, a lower immune score indicates a greater likelihood of effectiveness of a flavivirus vaccine in the subject. In some embodiments, a lower immune score indicates a lesser suitability to receive a flavivirus vaccine. In some embodiments, a lower immune score indicates a greater suitability to receive a flavivirus vaccine. In some embodiments, a lower immune score indicates a lower likelihood of effectiveness of a flavivirus vaccine. In some embodiments, a lower immune score indicates a lower likelihood of effectiveness of a flavivirus vaccine in the subject. In some embodiments, a lower immune score indicates a greater likelihood of effectiveness of a flavivirus vaccine. In some embodiments, a lower immune score indicates a greater likelihood of effectiveness of a flavivirus vaccine in the subject.

In some embodiments, a higher immune score indicates a greater risk of developing ADE. In some embodiments, a lower immune score indicates a lesser risk of developing ADE. In some embodiments, a higher immune score indicates a greater risk of a vaccine causing development of ADE. In some embodiments, development of ADE is in a subject. In some embodiments, a lower immune score indicates a lesser risk of developing ADE. In some embodiments, a lower immune score indicates a lesser risk of a vaccine causing development of ADE. In some embodiments, a higher immune score indicates the presence of a higher number of cross-reactive antibodies in a sample. In some embodiments, the presence of a higher number is an overexpression. In some embodiments, a lower immune score indicates the presence of a lower number of cross-reactive antibodies in a sample. In some embodiments, the presence of a lower number is a depletion. In some embodiments, cross-reactive antibodies are cross-reactive to a flavivirus. In some embodiments, cross-reactive antibodies are cross-reactive to a plurality of flaviviruses. In some embodiments, the flavivirus to which the antibodies are cross-reactive is the flavivirus for which there is an increased risk of ADE. In some embodiments, a higher immune score indicates a previous flavivirus infection. In some embodiments, a higher immune score indicates a previous flavivirus vaccination. In some embodiments the probes bound that result in the higher immune score indicate the particular flavivirus that caused the infection or that was vaccinated against.

In some embodiments, an immune score above a predetermined threshold indicates the subject is suitable to receive a flavivirus vaccination. In some embodiments, an immune score below a predetermined threshold indicates the subject is suitable to receive a flavivirus vaccination. In some embodiments, an immune score above a predetermined threshold indicates a flavivirus vaccine is likely to be effective. In some embodiments, an immune score below a predetermined threshold indicates a flavivirus vaccine is likely to be effective. In some embodiments, an immune score above a predetermined threshold indicates a flavivirus vaccine is likely to be effective in the subject. In some embodiments, an immune score below a predetermined threshold indicates the subject is suitable to receive a flavivirus vaccination. In some embodiments, an immune score below a predetermined threshold indicates a flavivirus vaccine is likely to be effective. In some embodiments, an immune score below a predetermined threshold indicates a flavivirus vaccine is likely to be effective in the subject. In some embodiments, an immune score below a predetermined threshold indicates a flavivirus vaccine is unlikely to be effective. In some embodiments, an immune score below a predetermined threshold indicates a flavivirus vaccine is unlikely to be effective in the subject. In some embodiments, likely comprises a chance of occurring of at least 50, 60, 70, 75, 80, 85, 90, 95, 97, 99 or 100%. Each possibility represents a separate embodiment of the invention. In some embodiments, unlikely comprises a chance of occurring of at most 1, 5, 10, 15, 20, 25, 30, 40 or 50%. Each possibility represents a separate embodiment of the invention.

In some embodiments, the method further comprises contacting immune cells with the flavivirus vaccine. In some embodiments, the contacting is done in vitro. In some embodiments, in vitro is in cell culture. In some embodiments, the immune cells are T cells. In some embodiments, the immune cells are B cells. In some embodiments, the immune cells are a mixed lymphocyte reaction. In some embodiments, the immune cells are peripheral blood mononuclear cells PBMCs. Contacting the immune cells with the vaccine can be done in any way known in the art. In some embodiments, the vaccine is added to the culture. In some embodiments, the vaccine is transferred into the cytosol of the immune cells. Methods of transfer such as transfection, nucleofection, and viral transfer (lentiviral etc.) are known in the art and any such method may be used.

In some embodiments, the immune cells are incubated for at least 1, 2, 3, 4, 5, 6, 7, 8, 9, 10, 11, 12, 13 or 14 days after contact with the flavivirus vaccine. Each possibility represents a separate embodiment of the invention. In some embodiments, the immune cells are incubated for a sufficient time to produce antibodies against a flavivirus. In some embodiments, media from the immune cells is the solution. In some embodiments, antibodies are isolated from the media. In some embodiments, proteins are isolated from the media.

As used herein, the term “about” when combined with a value refers to plus and minus 10% of the reference value. For example, a length of about 1000 nanometers (nm) refers to a length of 1000 nm+-100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Methods and Materials

Antigen microarray design and fabrication: Three types of antigens were spotted on the microarrays: whole viruses, recombinant proteins and peptides. Whole viruses were spotted as VLPs, cell lysate and inactivated virus. Antigens were printed on Hydrogel coated glass slides (SCHOTT, NEXTERION® Slide H) using a unique automated liquid dispensing array spotter (Scienion, sciFLEXARRAYER SX) in 16 array format. All antigens were spotted in triplicate. Viruses, proteins and peptides were also spotted as serial dilutions.

Recombinant proteins: Envelope and NS1 proteins were printed (see Table 1). The proteins had a great than 90% purity. Final concentration of 16.25 ug/ml protein in 0.0025%-0.01% triton X-100 or commercial spotting buffer (Scienion) were used for spotting. Diluted protein was stored in Genetix 384 plates in a −80-degree freezer. Synthetic peptides were spotted in 10-30% DMSO and 0.0025% triton X-100. Viruses were spotted in PBS or 0.0025% triton X-100.

Magnitude and breadth of summary statistics: The antibody profile generated by the antigen microarrays is a multidimensional measurement of the antibody responses to a large set of antigens. To compare the antibody responses of each sample as measured by these antigen microarrays across time and groups, the magnitude and breadth of responses to a given set of antigens was defined. The normalized array measurements was denoted by x_{i,p,ap} where: i—subject, =1, . . . , N; p—pathogen (flavivirus type), p=1, . . . , P; aₚ—antigen from pathogen, =1, . . . , Nₚ. zᵢ—denotes treatment assignment/group (vaccine/placebo, infected/vaccinated) of subject i. yᵢ—denotes outcome of subject i (vaccine-induced Ab titer/infection status/disease status). The observed data for each subject are (zᵢ, yᵢ, z_{i,p,ap}), for i=1, . . . , N; p=1, . . . , P; and aₚ=1, . . . , Nₚ. Fora given subject the ‘breadth’ and ‘magnitude’ of responses to each pathogen were defined as follows:

1.

$${m}_{i , p} = {\sum }_{{a}_{p} = 1}^{Np} {x}_{i , p , {a}_{p}}$$

—denotes the magnitude of responses to all antigens of pathogen.

2.

$${b}_{i , p} = {\sum }_{{a}_{p} = 1}^{Np} I ( {x}_{i , p , {a}_{p}} > 0 )$$

—denotes breadth of response to antigens from pathogen. The following data representations was considered for use in our statistical models:
1. x_{i,p,ap}—normalized array measurements.
2. d_{i,p}=f(i,p|a_{p,i}, . . . , a_{PN}_{_{P}})→[0,1]—where f ( ) is some function of the antigen measurements for pathogen p that maps to the binary indicator of whether the subject i has a detectable antibody response to pathogen: e.g. f(i,p|a_{p,i}, . . . , a_{PN}_{_{P}})=l(b_{i,p}) is 1 if a response is detected to at least one antigen from pathogenp, and 0 otherwise.
3. mᵢ=Σₚ₌₁^{P}m_{i,p}—denotes the magnitude of responses to all antigens.
4. bᵢ=Σᵢ₌₁^{P}d_{i,p}—denotes the breadth of responses to pathogens. DONE

Hybridization of antigen microarrays. Human serum samples were diluted 1:500-1:3000 for measuring anti-human IgG and 1:100-1:300 for measuring anti-human IaA (depending on the microarray format) in a hybridization buffer that contained 1% BSA in 0.025% PBST (0.025% tween-20 in PBS). Swab samples were diluted 1:2-1:20 for anti-human IgA. The spotted slides were blocked by a 1-hour incubation on a rocker at room temperature (RT) with a chemical blocking solution (50 mM ethanolamine, 50 mM borate, pH 9.0). After blocking the slides were washed twice with 0.05% PBST, twice with PBS and once with double-deionized water (DDW) (each wash—3 min on the rocker) and dried by centrifugation at RT for 5 minutes at 800g. Then the microarrays were hybridized with the diluted serum samples in divided trays for 2h at RT. Following washings as described above, the microarrays were incubated for 45 min with Alexa Fluor 647 labeled polyclonal anti-human IgG antibody at 1:1000 dilution (Jackson ImmunoResearch cat# 709-605-149), Alexa Fluor 488-conjugated polyclonal anti-human IgA antibody at 1:6000 dilution (Jackson ImmunoResearch cat# 109-545-011) or Alexa Fluor 647-conjugated polyclonal anti-human IgA antibody at 1:6000 dilution (Jackson ImmunoResearch cat# 109-605-011). The secondary antibodies were diluted in 1% BSA in 0.025% PB ST. To detect bound antibodies, slides were scanned on a four-laser GenePix 4400A scanner (Molecular Devices).

Measuring Antibody-dependent enhancement (ADE) of ZIKV infection by Flow cytometry. Antibody-dependent enhancement (ADE) of ZIKV infection was measured using a flow cytometry-based assay. Serial dilutions of serum were mixed with ZIKV (2 MOI), incubated for 1 hour at 37° C. and added to U937 cells (6 x 104) in 96 well U bottom plates in RPMI 1640 media supplemented with 10% FBS, 2mM L-glutamine, and 10U/ml penicillin and 10 μg/m1 streptomycin. After 2 days, cells were centrifuged and incubated on ice for 30 min with 7AAD viability dye. Cells were washed, fixed and permeabilized using True-Nuclear™ Transcription Factor Fixation/Permeabilization Buffer Set. Intracellular expression of Flavivirus group antigen was assayed by staining using monoclonal 4G2 antibody conjugated to AlexaFlour 467 (Anti-Flavivirus Group Antigen Antibody, clone D1-4G2-4-15, cat# MAB10216, Merck). Flow cytometry was performed with a Guava® easyCyte™ System and analyzed with Guava Soft version 3.3.

### Example 1: TBE Vaccination or Infection in Humans or Mice can Induce ADE of ZIKV Infection

In order to solve the question of whether previous infection or vaccination with one flavivirus can induce ADE for a different flavivirus, two cohorts of human subjects that were vaccinated or infected with tick-borne encephalitis virus (TBE) were investigated. TBE is tick transmitted and is routinely vaccinated against in many norther European countries such as Russia, Germany, Austria and Switzerland for example. Five licensed vaccines exist, FSME-IMMUN, Encepur, TBE-Moscow, EnceVir, and TBE-vaccine (China). Subjects often receive multiple boosters of the vaccine, as for example 58% of the population of Austria has been vaccinated at least 3 times.

A Siberian cohort of vaccinated, infected and control subjects was used. In the Siberian cohort 77 subjects were vaccinated with the Russian vaccine, 30 subjects were naive to TBE and 37 had previously suffered an acute infection. Zika ADE was measured in each individual using the plaque assay or intracellular staining (ICS), which are the canonical characterizations of ADE in vitro. Serum sample from a single subject (AD) who previously had TBE vaccination and had also been infected with Dengue virus type 2 was used as a positive control. The serum of subject AD was found to have a high ADE of Zika virus infection in vitro.

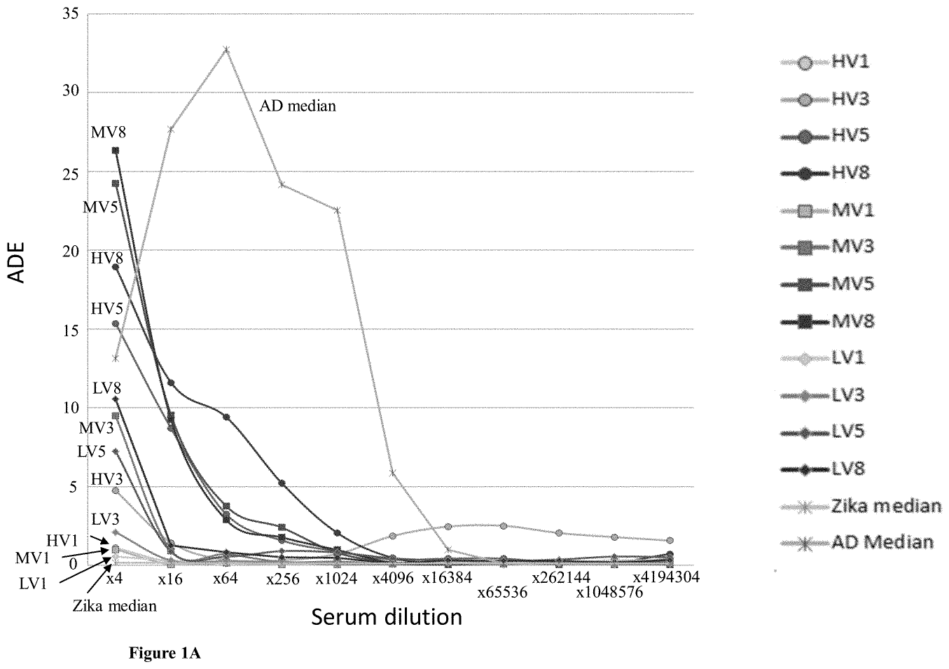

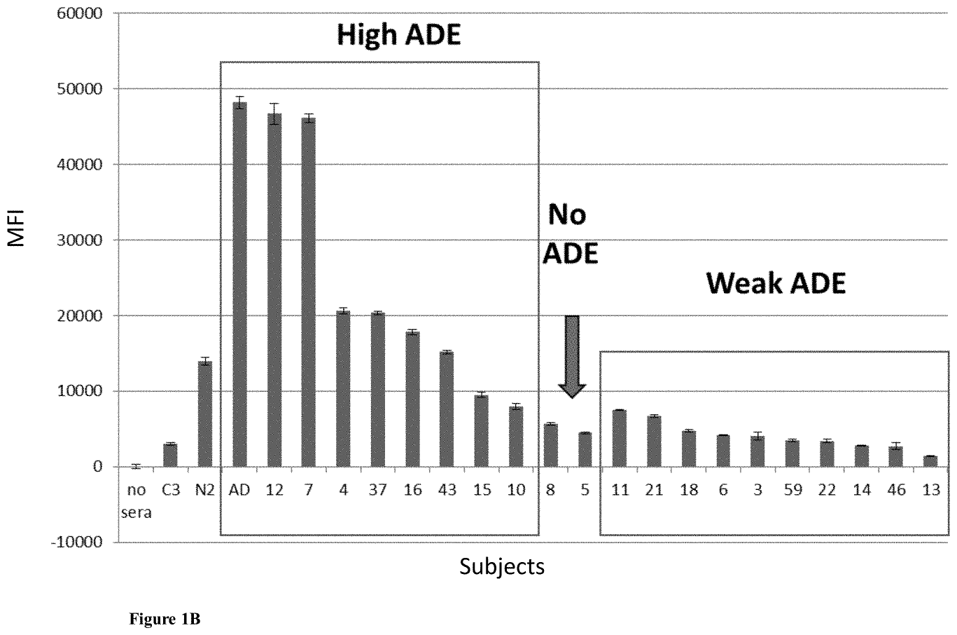

A second human cohort from Switzerland (Swiss cohort) included sera samples from 137 humans that were vaccinated 3 times (at weeks 0, 4 and 24) with a TBE vaccine. Serum samples were collected at baseline (before vaccination, visit 1), 4 weeks post primer (visits 3, 4 weeks after first vaccination), 4 weeks post first booster (visit 5, week 8) and 4 weeks post second booster (visit 8, week 28), and hybridized with the array of invention. After vaccination, TBE IgG titers were measured by ELISA for all the subjects. Three subgroups of subjects (n=15-17 per group) with the highest (H), Lowest (L) and a medium (M) IgG titer to TBE were selected. As can be seen in FIG. 1A, at the later time points, visits 5 and 8, subjects showed increased ADE. Specifically, the high and medium group of subjects showed the highest ADE of Zika virus (ZIKV) infection (measured in vitro). This indicates that vaccination against TBE can induce ADE for Zika, confirming the cross flavivirus risk of vaccination. Individuals from the high and medium anti-TBE titer groups presented higher ZIKV ADE compared with individuals from the low group.

In both cohorts, sera from only some of the individuals that were exposed to TBE by infection or vaccination induced ADE of Zika virus in vitro. Of note, simple measurement of the total IgG titer to Zika virus by ELISA could not accurately identify the individuals at risk for ZIKV ADE. For example, in TBE vaccinated individuals from the Siberian cohort, although there was a general trend of correlation between antibody titers to ZIKV (as measured by ELISA) and ADE, the relationship was not always predictive (FIG. 1B, showing representative results from the Siberian cohort). Some individuals with equal antibodies levels had high amounts of ADE, some had only weak ADE and some showed no ADE whatsoever (FIG. 1B, see subjects, 10, 11 and 8 for example). This indicates that total IgG levels alone are not sufficient for predicting who will develop ADE.

To further test whether TBE vaccination can induce ZIKV ADE in vivo, mice were vaccinated with the Siberian TBE vaccine. Wild-type Balb/C mice that were not immune compromised were vaccinated subcutaneously twice with the Russian TBE vaccine (100 ul) to induce antibody production. A control group was sham-injected with PBS. Mice were injected at days 0 and 21 of the experiment. All mice were then challenged with Zika virus (intraperitoneal injection of various doses, see Table 3) and monitored for Zika symptoms. Blood samples were also collected 7 days after the Zika challenge and PCR was performed for Zika RNA. As expected, none of the mice developed Zika symptoms. As these are healthy, wild-type, non-immunocompromised mice, the virus should be eradicated within a week. However, when PCR was run, 5 of the 9 TBE vaccinated mice were found to still be positive for Zika RNA, indicating the virus had not been eradicated from the mice (see Table 3). All of the control mice (no vaccination) were negative for Zika RNA. This indicates that in the mice the TBE vaccination exacerbated the Zika infection, confirming that ADE across flaviviruses occurs.

TABLE 3
Mice summary results

| | ZIKV dose | | #mice | |
|---|---|---|---|---|
| Vaccination | (×10⁶) | #mice | PCR positive | % positive |
| TBE | 22.8 | 3 | 2 | 67% |
| PBS | 22.8 | 3 | 0 | 0% |
| TBE | 1.5 | 6 | 3 | 50% |
| PBS | 1.5 | 6 | 0 | 0% |

### Example 2: Different Antibody Repertoires in TBE Infected and TBE Vaccinated Humans

Sera from the subjects of the two human cohorts were hybridized with the arrays of the invention and antibody binding was detected by fluorescent secondary antibodies and a laser scanner reader.

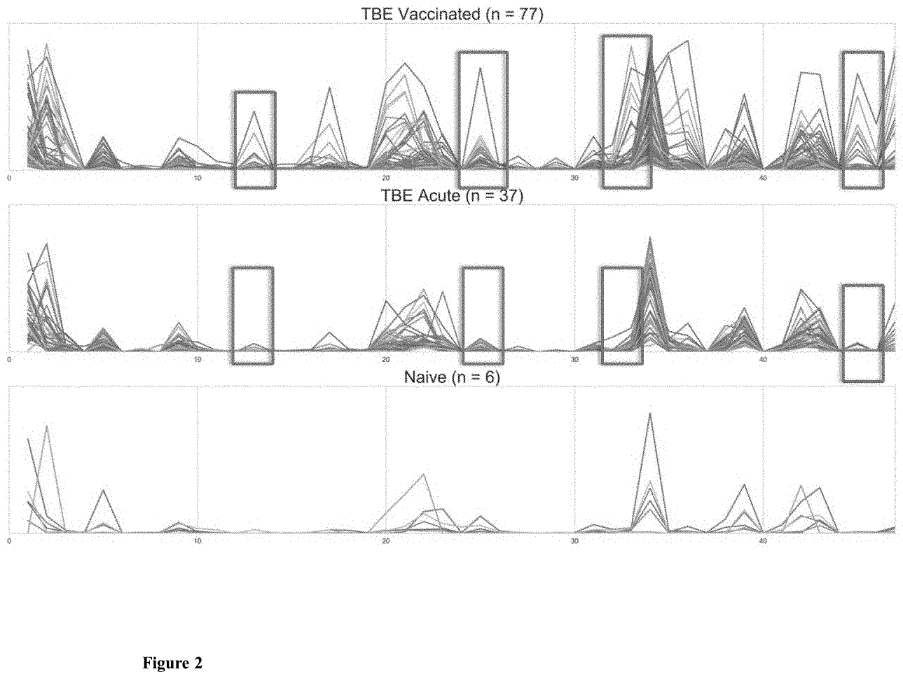

FIG. 2 shows a plot of the antibody repertoire of individuals from the Siberian cohort post vaccination or post infection. Each line represents a single subject, and each peak is the binding detected to a specific probe on the array of the invention. Only Zika virus envelope peptide antigens are shown in FIG. 2. As is evident, there is a great deal of variability from subject to subject, and even more so between the vaccinated subject, infected subject and naive subjects. A subset of peptides was identified that could distinguish between infected and vaccinated individuals.

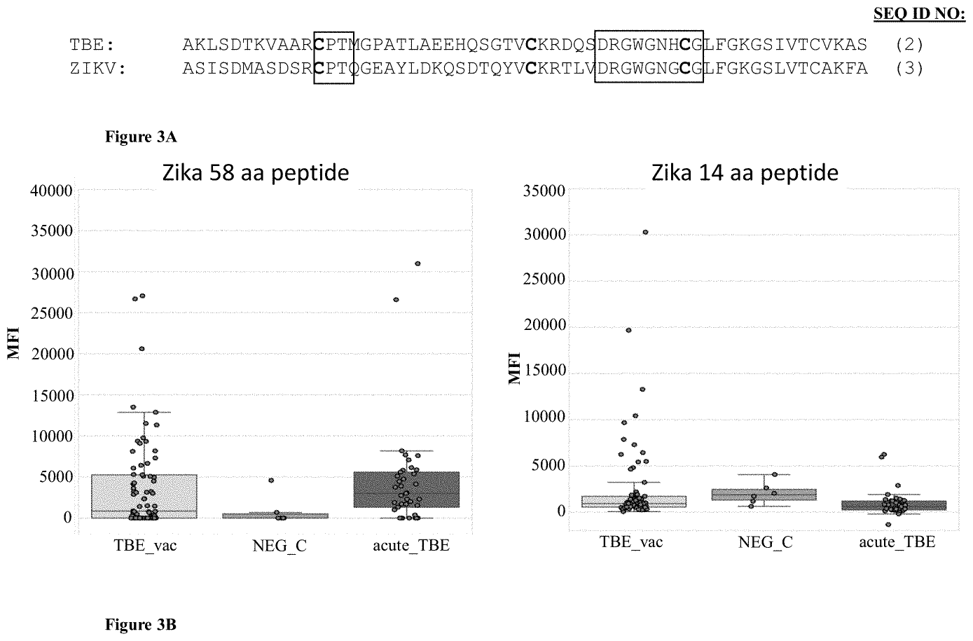

The fusion loop region of flaviviruses is well studied, and it is highly conserved across flaviviruses (FIG. 3A). FIG. 3B shows the binding of antibodies from the three groups of the Siberian cohort (TBE vaccinated, TBE infected, naive to flaviviruses) to a 58 amino acid peptide representing the entire fusion loop (left graph) and to a shorter 14 amino acid peptide in the most highly conserved region of the fusion loop (right graph, see black box in FIG. 3A). While both TBE infected and TBE vaccinated subjects can develop antibodies to the Zika fusion loop peptides, only part of the individuals in each group developed such antibodies.

### Example 3: Dengue Titers Generated by TBE Vaccine were Identified by Whole Virus Antigens

The serum samples of the individuals from the high, medium and low groups of the Swiss cohort (grouped based on anti-TBE IgG titers) which presented different ADE levels were also hybridized to the arrays of the invention (FIG. 1A). These subjects were considered at two time points: visit 1 before the first vaccination (week 0) and visit 8 after the third vaccination (4 weeks after the second booster, week 28). Once again, a single patient (AD) who was known to have been both infected with Dengue type 2 and TBE vaccinated was used as a positive control. The results of the array analysis for these subjects were investigated.

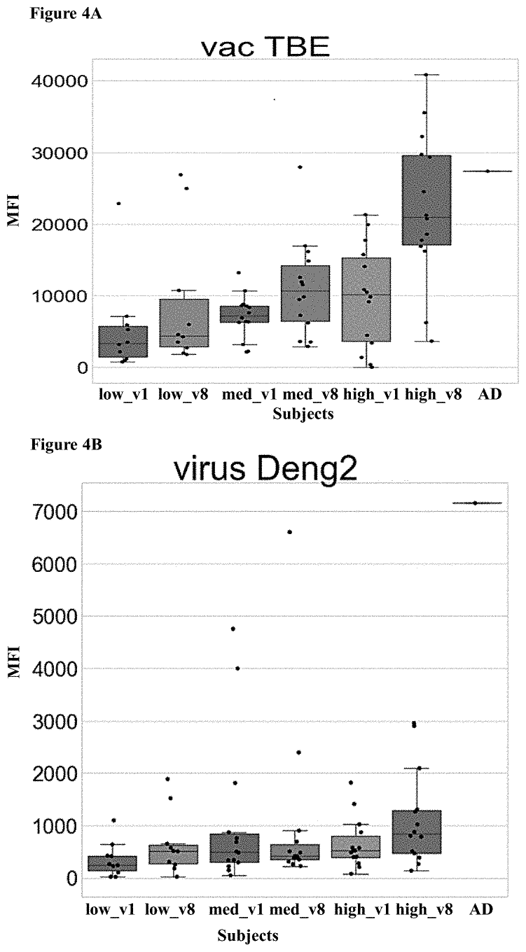

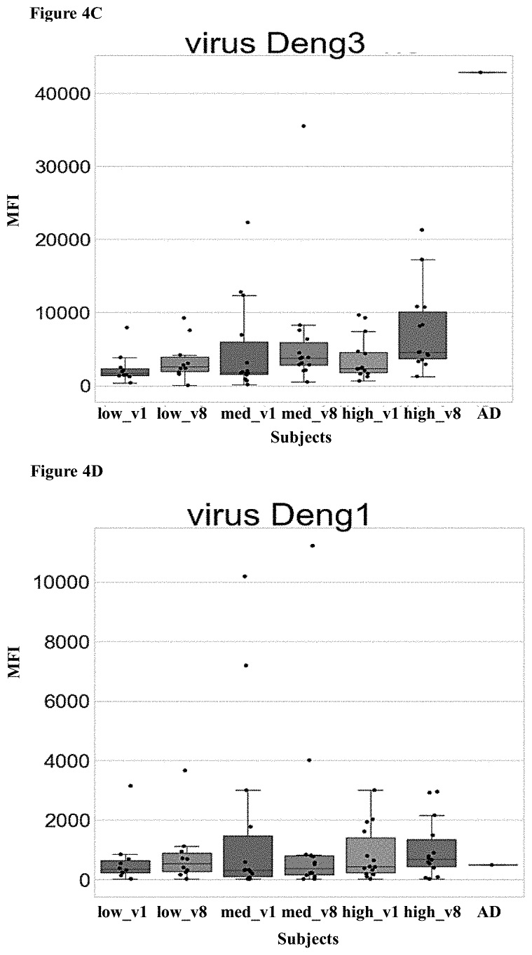

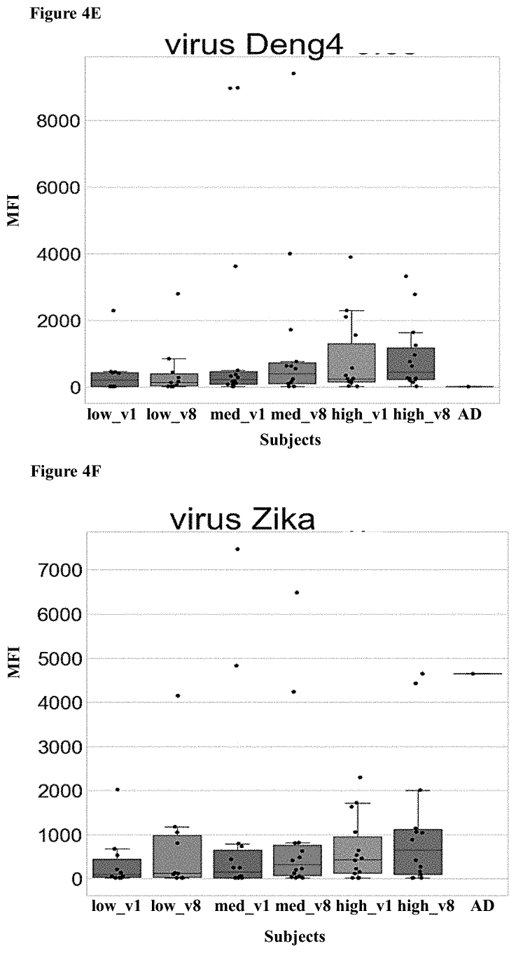

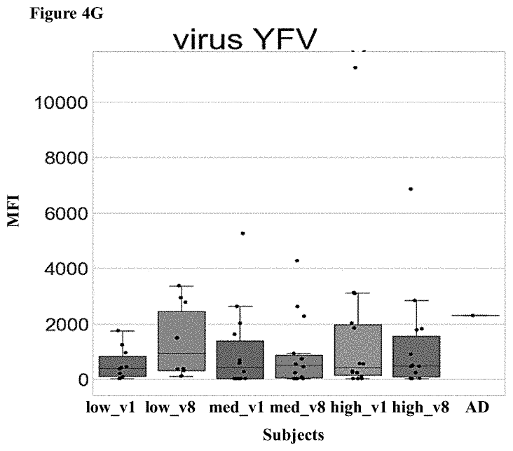

When binding of antibodies to TBE vaccine probes on the array was measured, as expected an increase in IgG binding from baseline (pre-vaccination, v1) to post-vaccination (v8) was observed for all three groups (FIG. 4A). The differences between the groups correlated with the ELISA results (H>M>L). While most of the subjects in a group clustered together, as can be seen in the box and whisker plot, there were several outliers in all groups with significantly higher titer levels. Subject AD had high titer levels comparable to the highest group after the booster vaccination.

Next, binding of antibodies to Dengue type 2 probes on the array was examined (FIG. 4B). Subject AD has high levels of Dengue 2 binding antibodies (>7000 MFI). This is to be expected as the subject had a previous Dengue 2 infection. Unexpectedly, several subjects from the three groups of the Swiss cohort had elevated anti-Dengue 2 antibodies, with one subject having levels almost as high as subject AD. Subjects with an WI of over 2000 were considered to have a positive response, and such individuals were observed after both the first and second vaccinations. Similar results were observed for antibodies against Dengue type 3 (FIG. 4C). Again, several individuals had antibody levels comparable to subject AD.

When Dengue type 1 (FIG. 4D) and Dengue type 4 (FIG. 4E) were investigated, a similar pattern was observed. There were several individuals with unexpectedly high cross-reactive antibodies. Of note, subject AD had almost no cross-reactive antibodies to Dengue type 1 and 4, indicating that vaccination/infection by a more distant flavivirus evolutionarily speaking can have an as potent or even more potent effect on ADE.

Antibody cross-reactivity to two other flaviviruses were also checked: Zika (FIG. 4F), and Yellow Fever virus (FIG. 4G). Certain individuals were again found to have highly cross-reactive antibodies present in their samples. These results show that the array can be used to predict individuals that are at increased risk for developing ADE for various flaviviruses upon future infection.

### Example 4: Recombinant Proteins and Short Peptides on the Array

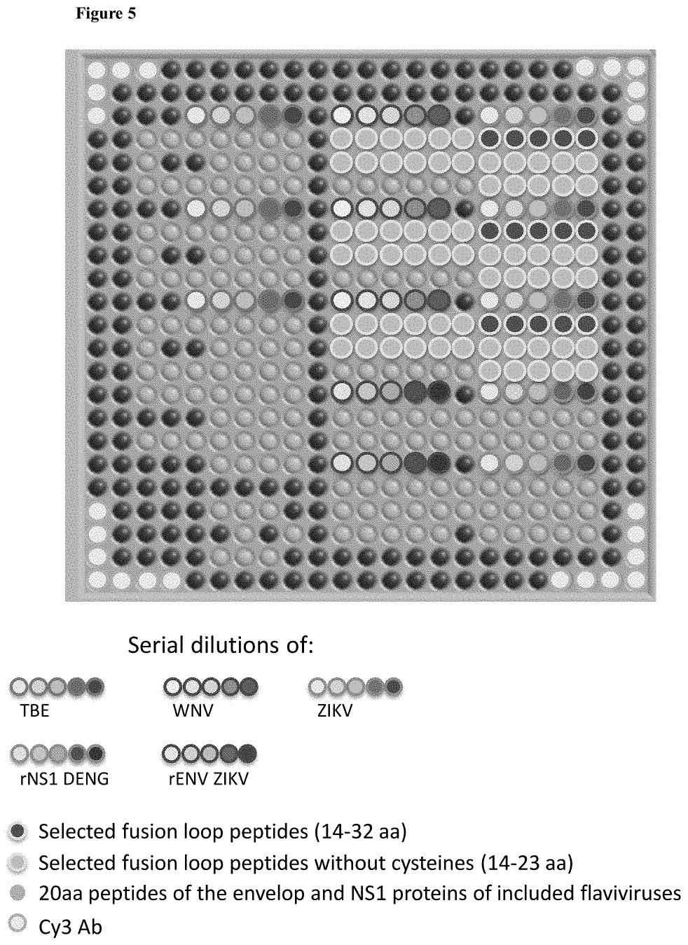

The array of the invention included not only full viruses which contain 3D, or conformational, epitopes, but also recombinant viral proteins and short viral peptides. The viral proteins are expected to be at least partially folded and thus would also contain conformational epitopes, while short peptides (14-20 amino acids) would be expected to remain mostly linear and thus provide linear epitopes. The longer peptides of the envelope fusion loop (32, 58 aa), which contain at least two cysteine residues, may be folded to create a conformational loop. To distinguish between antibodies to linear and 3D epitopes of the fusion loop, the array also included mutated loop peptides, in which the cysteine residues were replaced by methionine residues. A schematic diagram of one block from an exemplary array is provided in FIG. 5. The array contains inactivated viruses (TBE, WNV, ZIKV for example), full recombinant proteins (rNS1, rENV for example) and short peptides, provided as serial dilutions and in triplicates.

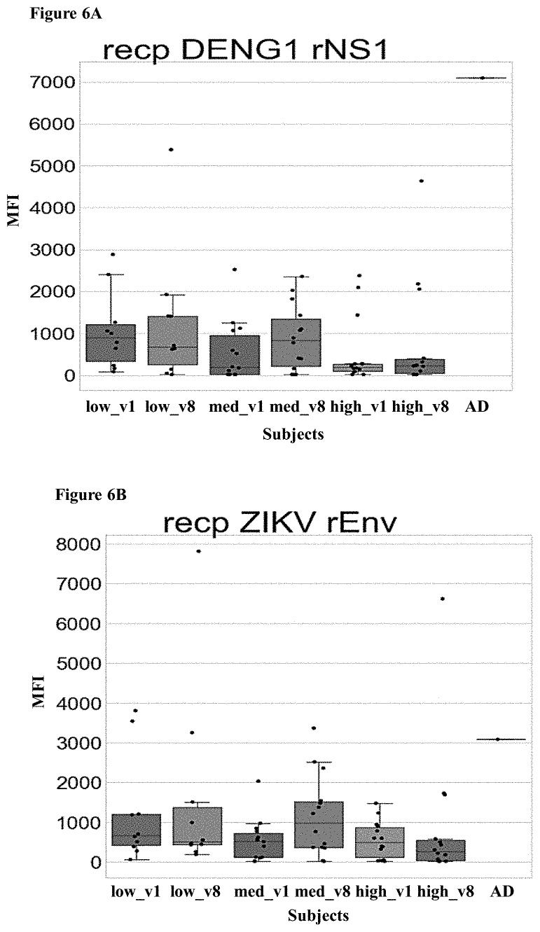

The effectiveness of the full virus has been shown hereinabove, however, recombinant proteins and short peptides were also found to be effective. Subject were found with antibodies that bound to the recombinant Dengue 1 NS1 proteins (FIG. 6A), similar to what was observed for the whole virus (FIG. 4D). Interestingly, though the control subject AD had low antibody levels against Dengue 1 whole virus, this subject showed strong antibody expression against Dengue 1 NS1. Subjects with cross-reactive antibodies to the recombinant Zika Envelope protein (Env) were also identified (FIG. 6B).

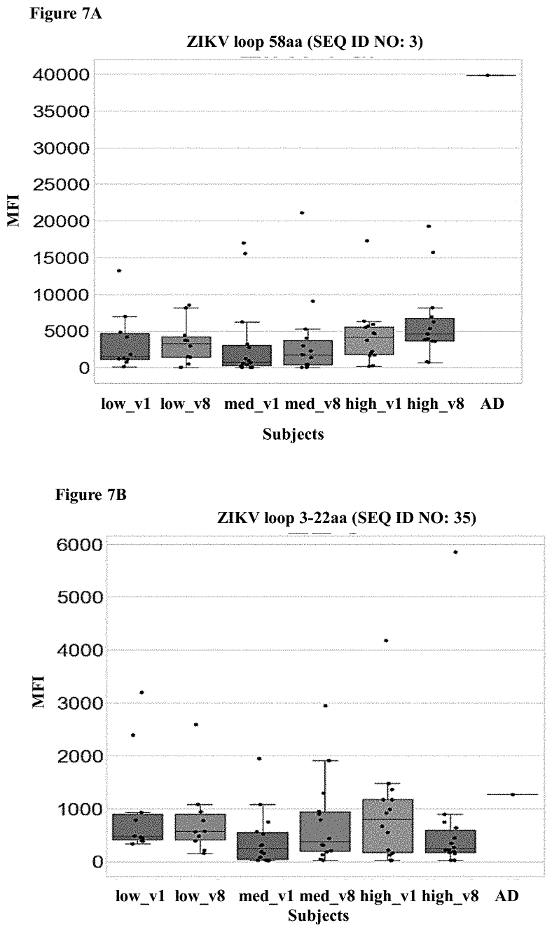

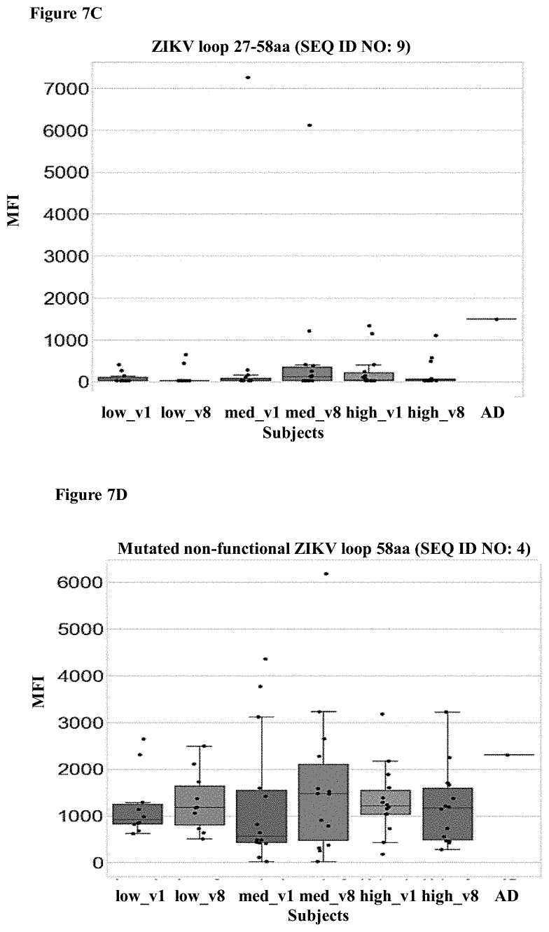

As before the 58-amino acid region of the fusion loop was also examined (ASISDMASDSRCPTQGEAYLDKQSDTQYVCKRTLVDRGWGNGCGLFGKGSLVTC AKFA, SEQ ID NO: 3). There is a high level of conservation in the fusion loop between various flaviviruses (see FIG. 3A for example). A robust cross-reactivity for the Zika full fusion loop was indeed observed (FIG. 7A). FIG. 7A shows this cross-reactivity and it is important to note that the Y axis of this figure is significantly higher than what has been observed previously. If an MFI of 2000 is used as a positive threshold, as had been suggested previously, then a far greater percentage of the TBE vaccinated cohort is found to be cross-reactive. Indeed, in the high subpopulation after vaccination, more than 50 percent of the subjects were positive for cross-reactivity. Shorter peptides covering amino acids 3-22 (ISDMASDSRCPTQGEAYLDK, SEQ ID NO: 35, FIG. 7B) and 27-58 (QYVCKRTLVDRGWGNGCGLFGKGSLVTCAKF, SEQ ID NO: 9, FIG. 7C) produced significantly reduced binding, close to an order of magnitude reduced, suggesting that a large fraction of the antibodies to the fusion loop target conformational loop epitopes. However, the short loop peptides were also effective in capturing subjects with cross-reactive antibodies. It was also observed that functional peptides were required for optimal antibody binding. The fusion loop of Zika was mutated at four residues (ASISDMASDSRCPAGGEAYLDKQSDTQYVCKRTLVDRGRGNGCGRFGKGSLVTC AKFA, SEQ ID NO: 4) that have been reported to abolish functionality of the loop. Antibody binding to this mutant sequence was reduced by close to an order of magnitude (FIG. 7D), indicating that functional peptides are advantageous for antibody detection.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An array comprising a plurality of probes each immobilized at a discrete location on said array, wherein said plurality of probes comprises a probe from a first flavivirus and a probe from a second flavivirus.

2. The array of claim 1, wherein said plurality of probes comprises at least two probes from each flavivirus.

3. The array of claim 1, wherein said flavivirus is selected from the group consisting of: Zika virus, Dengue virus type 1, Dengue virus type 2, Dengue virus type 3, Dengue virus type 4, West Nile virus, Japanese encephalitis virus, Tick-borne encephalitis virus, Louping ill virus, Omsk hemorrhagic fever virus, Powassan virus, Apoi virus, Yokose virus, Yellow fever virus, Rocio virus, Ilheus Virus, Bagaza virus, St. Louis encephalitis virus, Murray Valley encephalitis virus, Alfuy Virus and Usutu virus.

4. (canceled)

5. The array of claim 3, wherein said first and second flaviviruses are Zika virus and Tick-borne encephalitis virus.

6. The array of claim 3, wherein said array comprises a plurality of probes from Dengue virus type 1, Dengue virus type 2, Dengue virus type 3, and Dengue virus type 4.

7. The array of claim 1, wherein said plurality of probes are selected from a whole virus, a lysed virus, a virus-like particle (VLP), a whole recombinant protein and a peptide.

8. The array of claim 1, wherein said plurality of probes comprises:
a. a peptide probe from each of said flaviviruses;
b. a peptide probe from a viral NS1 protein from each of said flaviviruses,
c. a peptide probe from each of said flaviviruses wherein said peptide probe comprises between 10 and 60 consecutive amino acids from a flavivirus protein; or
d. a combination thereof.

9. The array of claim 8, wherein said plurality of probes comprises a peptide probe from a viral envelope protein from each of said flaviviruses or from a fusion loop region from each of said flaviviruses.

10. (canceled)

11. (canceled)

12. (canceled)

13. The array of claim 1, wherein said plurality of probes comprises a recombinant protein from each of said flaviviruses, an inactivated form of each of said flaviviruses, a virus-like particle (VLP) of each of said flaviviruses, lysate from a cell infected by each of said flavivirus, or a combination thereof.

14. (canceled)

15. (canceled)

16. (canceled)

17. The array of claim 1, comprising serial dilutions of at least one probe, wherein each dilution is immobilized at a discrete location on said array.

18. The array of claim 1, wherein said plurality of probes is selected from Table 1, Table 2 or both.

19. (canceled)

20. (canceled)

21. (canceled)

22. (canceled)

23. (canceled)

24. A method of measuring cross-reactive antibodies to a flavivirus in a subject in need thereof, the method comprising
a. providing a biological sample from said subject comprising antibodies;
b. contacting said sample to an array of claim 1 in conditions sufficient for antibody binding to said probes; and
c. detecting the binding of said antibodies to discrete locations on said array indicating the presence in said sample of antibodies to probes located at said detected discrete locations;
thereby measuring cross-reactive antibodies to a flavivirus.

25. The method of claim 24, wherein said subject has previously been vaccinated against a flavivirus or previously been infected by a flavivirus, wherein said biological sample is a peripheral blood sample, a plasma sample or a serum sample or both.

26. The method of claim 24, wherein said flavivirus is selected from the group consisting of: Zika virus, Dengue virus type 1, Dengue virus type 2, Dengue virus type 3, Dengue virus type 4, West Nile virus, Japanese encephalitis virus, Tick-borne encephalitis virus, Louping ill virus, Omsk hemorrhagic fever virus, Powassan virus, Apoi virus, Yokose virus, Yellow fever virus, Rocio virus, Ilheus Virus, Bagaza virus, St. Louis encephalitis virus, Murray Valley encephalitis virus, Alfuy Virus and Usutu virus.

27. (canceled)

28. The method of claim 24, wherein said detecting comprises contacting said array with bound antibodies with labeled secondary antibodies against said antibodies in said biological sample, and optionally scanning said array with a detector configured to detect said labeled secondary antibodies and producing an output of the discrete locations where antibody was detected.

29. (canceled)

30. The method of claim 24, wherein said method is a method of assessing a risk of developing ADE upon infection of said subject by a flavivirus and further comprising:
d. generating a flavivirus immune score from said detected binding, wherein the magnitude of said immune score is proportional to the risk of developing ADE.

31. The method of claim 30, wherein a higher immune score indicates a greater risk of developing ADE upon flavivirus infection, and wherein a lower immune score indicates a lesser risk of developing ADE upon flavivirus infection or wherein an immune score above a predetermined threshold indicates the subject is at an increased risk of developing ADE upon flavivirus infection.

32. (canceled)

33. A method of predicting a risk of future ADE induction due to vaccination by a flavivirus vaccine, the method comprising:
a. providing a solution comprising antibodies from immune cells contacted by said flavivirus vaccine;
b. contacting said solution to an array of claim 1 in conditions sufficient for antibody binding to said probes;
c. detecting the binding of said antibodies to discrete locations on said array indicating the presence in said solution of antibodies to probes located at said detected discrete locations; and
d. generating a flavivirus immune score from said detected binding, wherein the magnitude of said flavivirus immune score is proportional to the risk of future ADE induction due to vaccination by said flavivirus vaccine;
thereby predicting the risk of future ADE induction due to vaccination by a flavivirus vaccine.

34. A kit comprising the array of claim 1, and a labeled secondary antibody configured for detection of antibodies bound to said array.

35. A system comprising the array of claim 1, and a detector configured to detect binding of antibodies to probes immobilized on said array, optionally wherein said detector is configured to detect labeled secondary antibodies.

36. (canceled)
